(19) United States
(12) Patent Application Publication
(10) Pub. No.: US 2024/0315665 A1
(43) Pub. Date: Sep. 26, 2024

(54) METHOD AND SYSTEM FOR OBSERVING BRAIN STATES WITH FUNCTIONAL ULTRASOUND IMAGING AND A SONOLUCENT INTERFACE

(71) Applicants: California Institute of Technology, Pasadena, CA (US); University of Southern California, Los Angeles, CA (US)

(72) Inventors: Sumner L. Norman, Pasadena, CA (US); Claire Rabut, Pasadena, CA (US); Whitney S. Griggs, Pasadena, CA (US); Richard A. Andersen, Pasadena, CA (US); Mikhail G. Shapiro, Pasadena, CA (US); Charles Liu, Los Angeles, CA (US)

(21) Appl. No.: 18/734,823

(22) Filed: Jun. 5, 2024

Related U.S. Application Data
(63) Continuation-in-part of application No. 18/505,827, filed on Nov. 9, 2023.
(60) Provisional application No. 63/424,235, filed on Nov. 10, 2022.
(60) Provisional application No. 63/471,061, filed on Jun. 5, 2023.

(51) Int. Cl. A61B 8/00; A61F 2/28

(52) U.S. Cl. CPC A61B 8/4209; A61F 2/2875

(57) ABSTRACT

A cranial implant that facilitates functional ultrasound imaging is disclosed. The example cranial implant replaces a section of a skull. The cranial implant in includes a support section shaped to replace a removed section of the skull. The implant includes a window of sonolucent material allowing functional ultrasound imaging through an ultrasound probe on the window. The window is shaped to allow access to a region of interest in the brain from the ultrasound probe.

### 1. PRIORITY CLAIM

This is a continuation in part of U.S. patent application Ser. No. 18/805,827 filed Nov. 9, 2023, which claims priority to and the benefit of U.S. Provisional Application No. 63/424,235, filed Nov. 10, 2022. This application also claims priority to and the benefit of U.S. Provisional Application No. 63/471,061, filed Jun. 5, 2023. The contents of these applications in their entirety are hereby incorporated by reference.

### 2. FEDERAL GOVERNMENT SUPPORT STATEMENT

The subject matter of this invention was made with government support under Grant Nos. EY032799 & NS123663 awarded by the National Institutes of Health. The government has certain rights in the invention.

### 3. TECHNICAL FIELD

The present disclosure relates to facilitation of functional ultrasound imaging, and specifically to a sonolucent skull material implant for a functional ultrasound imaging sensor system.

### 4. BACKGROUND

The following description includes information that may be useful in understanding the present invention. It is not an admission that any of the information provided herein is prior art or relevant to the presently claimed invention, or that any publication specifically or implicitly referenced is prior art.

Brain machine-interface (BMI) technologies communicate directly with the brain and can improve the quality of life of millions of patients with brain disorders. Motor BMIs are among the most powerful examples of BMI technology. Ongoing clinical trials of such BMIs implant microelectrode arrays into motor regions of tetraplegic participants. Movement intentions are decoded from recorded neural signals into command signals to control a computer cursor or a robotic limb. Clinical neural prosthetic systems enable paralyzed human participants to control external devices by: (a) transforming brain signals recorded from implanted electrode arrays into neural features; and (b) decoding neural features to predict the intent of the participant. However, these systems fail to deliver the precision, speed, degrees-of-freedom, and robustness of control enjoyed by motor-intact individuals. To enhance the overall performance of the BMI systems and to extend the lifetime of the implants, newer approaches for recovering functional information of the brain are necessary.

Brain-machine interfaces (BMIs) translate complex brain signals into computer commands and are a promising method to restore the capabilities of patients with paralysis. State-of-the-art BMIs have already been proven to function in limited clinical trials. However, these current BMIs require invasive electrode arrays that are inserted into the brain. Device degradation limits the longevity of BMI systems that rely on the implanted electrodes to typically around 5 years. Further, the implants only sample from small regions of the superficial cortex. Their field of view is small, restricting the number, and type, of applications possible. These are some of the factors limiting adoption of current BMI technology to a broader patient population.

Recording human brain activity is crucial for understanding normal and aberrant brain function and for developing effective brain-machine interfaces (BMIs). However, available recording methods are either highly invasive or have relatively low sensitivity. Functional magnetic resonance imaging (fMRI) accesses the whole brain but has limited sensitivity (requiring averaging) and spatiotemporal resolution. It additionally requires the participant to lie in a confined space and minimize movements, restricting the tasks they can perform. Other non-invasive methods, such as electroencephalography and functional near infrared spectroscopy, are affordable and portable. However, the signals they produce are limited by volume conduction or scattering effects, resulting in poor signal-to-noise ratios and limited ability to measure function in deep brain regions. Magnetoencephalography has good spatiotemporal resolution but is limited to cortical signals. Intracranial electroencephalography and electrocorticography have good temporal resolution and better spatial resolution but are highly invasive, requiring subdural implantation. Implanted microelectrode arrays set the gold standard in sensitivity and precision by recording the activity of individual neurons and local field potentials. However, these devices are highly invasive, requiring insertion into the brain. Moreover, they are difficult to scale across many brain regions and have a limited functional lifetime due to tissue reactions or breakdown of materials over time. To date, only severely impaired participants for whom the benefits outweigh the risk have used invasive recording technologies. There is a clear and distinct need for neurotechnologies that optimally balance the tradeoffs between invasiveness and performance.

Functional ultrasound imaging (fUSI) is an emerging technique that offers sensitive, large-scale, high-resolution neural imaging. Functional ultrasound imaging (fUSI) is an emerging neuroimaging technique that spans the gap between invasive and non-invasive methods. It represents a new platform with high sensitivity and extensive brain coverage, enabling a range of new pre-clinical and clinical applications. Based on power Doppler imaging, fUSI measures changes in cerebral blood volume (CBV) by detecting the backscattered echoes from red blood cells moving within its field of view (several cm). fUSI is spatially precise down to ˜100 μm with a framerate of up to 10 Hz, allowing it to sense the function of small populations of neurons. fUSI is minimally invasive and requires only removal or replacement of a small skull area in large organisms. fUSI does not intrude on brain tissue but instead sits outside the brain's protective dura mater and does not require the use of contrast agents. fUSI is non-radiative, portable, and proven across multiple animal models (rodents, ferrets, birds, non-human primates, and humans). In recent work, the intentions and goals of non-human primates have been decoded from fUSI data and subsequently fUSI was used as the basis for the first ultrasonic brain-machine interface (BMI).

An important direction of this research is the translation of fUSI-based neuroimaging and BMI for human participants. However, the skull bone attenuates and aberrates acoustic waves at high frequencies, substantially reducing signal sensitivity. As a result, most pre-clinical applications require a craniotomy, and the few human fUSI studies have required the skull to be removed or absent. These include intra-operative imaging during neurosurgery and recording through the anterior fontanelle window of newborns. Using fUSI to record brain activity in awake adults outside of an operating room is currently impossible. However, fUSI cannot be performed through adult human skull due to the skull attenuating the ultrasound signal underlying the standard fUSI methodology.

It is currently difficult and expensive to monitor anatomical and functional brain recovery following a cranioplasty. Behavioral assessments, such as Cognitive Status Examination, Mini-Mental State Examination, or Functional Independence Measure are commonly used to assess neuropsychological recovery following traumatic brain injuries but cannot identify specific sites of damage or track recovery at these anatomical locations. Less commonly, CT and/or MRI are used to assess anatomical and functional recovery. However, these methods possess low sensitivity/specificity for assessing brain recovery, are expensive (CT+MRI), and can add risk to the patient (CT).

Further, one of the most significant bottlenecks to human neuroscience research and the development of less invasive BMI systems is the limited access to human patients for obtaining neural activity data. The ability to measure fUSI signals from fully reconstructed, ambulatory adult humans has the potential to address this challenge, opening opportunities for advancements in these areas. Approximately 1.7 million people suffer from a severe traumatic brain injury each year in the United States.

Thus, the next generation of BMI technology requires an interface to access the brain to allow the evaluation of functional ultrasound imaging for BMI design. There is a further need for a reconstructive interface that may be non-intrusive to allow functional ultrasound imaging in adult humans.

### 5. SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

One example is a cranial implant replacing a section of a skull for functional ultrasound imaging. The cranial implant includes a support section shaped to replace a removed section of the skull. A window of sonolucent material in the support section allows functional ultrasound imaging through an ultrasound probe on the window. The window is shaped to allow access to a region of interest in the brain.

In another disclosed implementation of the example cranial implant, the window of the sonolucent material includes the entire support section. In another disclosed implementation, the sonolucent window is fabricated from one of polymethyl methacrylate (PMMA) or polyether ether ketone (PEEK). In another disclosed implementation, the sonolucent window is a titanium mesh. In another disclosed implementation, the sonolucent window is positioned above at least one of a primary motor cortex, a primary somatosensory cortex, or a posterior parietal cortex of the brain when the support section replaces the removed section of the skull. In another disclosed implementation, the sonolucent window has a thickness of between 1 and 10 mm. In another disclosed implementation, the sonolucent window is a subsection of the support section, and the sonolucent window has a thickness less than the thickness of the support section. In another disclosed implementation, the ultrasound probe is positioned near a Postcentral gyrus (poCG) and a Supramarginal gyrus (SMG) of the brain. In another disclosed implementation, the window is a parallelogram shape.

Another example is a method for producing a customized cranial implant for collecting functional ultrasound images. The method includes performing a craniectomy on a patient. The brain underneath a skull section removed through the craniotomy is imaged. A cranial implant is fabricated to replace the skull section removed through the craniotomy. A sonolucent window in the implant above a region of the brain based on the imaging is created to allow collection of functional ultrasound images via an ultrasound transducer probe.

In another disclosed implementation of the example method, the imaging is performed via either a magnetic resonance imaging system or a functional ultrasound system. In another disclosed implementation the window of the sonolucent material includes the entire support section. In another disclosed implementation the sonolucent window is fabricated from one of polymethyl methacrylate (PMMA) or polyether ether ketone (PEEK). In another disclosed implementation the sonolucent window is a titanium mesh. In another disclosed implementation the sonolucent window is positioned above at least one of a primary motor cortex, a primary somatosensory cortex, or a posterior parietal cortex of the brain. In another disclosed implementation the sonolucent window has a thickness of between 1 and 10 mm. In another disclosed implementation the sonolucent window is a subsection of the support section, and the sonolucent window has a thickness less than the thickness of the support section. In another disclosed implementation the ultrasound transducer probe is positioned near a Postcentral gyrus (poCG) and a Supramarginal gyrus (SMG) of the brain.

Another disclosed example is a functional ultrasound imaging system including an ultrasound probe having a transducer for functional ultrasound imaging. A cranial implant includes an imaging window constructed of sonolucent material. The functional ultrasound probe is positioned on the imaging window. A functional ultrasound controller is coupled to the ultrasound probe. The functional ultrasound controller takes functional ultrasound images of a brain via the ultrasound probe.

### 6. BRIEF DESCRIPTION OF DRAWINGS

In order to describe the manner in which the above-recited disclosure and its advantages and features can be obtained, a more particular description of the principles described above will be rendered by reference to specific examples illustrated in the appended drawings. These drawings depict only example aspects of the disclosure, and are therefore not to be considered as limiting of its scope. These principles are described and explained with additional specificity and detail through the use of the following drawings:

FIG. 1 shows an example brain machine interface system using an ultrasound implant, a trained decoder, and a control system for moving a cursor on a display;

FIG. 2A is a hardware diagram of an example test system used in conducting tests relating to the example trained BMI;

FIG. 2B is a perspective view of an ultrasonic transducer in the example test system in FIG. 2A:

FIG. 2C is a cross section of the ultrasonic transducer in the example test system in FIG. 2A;

FIG. 2D is a block diagram of the software components of the test system in FIG. 2A;

FIG. 3A is a set of images of brains of non-human primate test subjects taken from the test system in FIG. 2A;

FIG. 3B is a block diagram of a test for the example system in FIG. 2A that included memory guided saccade tasks;

FIG. 3C is a block diagram showing process of the collection of test data by the test system in FIG. 2A;

FIG. 3D is a block diagram of a test for the example system in FIG. 2A that included memory guided BMI tasks;

FIG. 3E is a diagram showing the test process for the memory-guided BMI task;

FIG. 4A is a set of results from a test using a two direction memory guided saccade task with no pretraining of the BMI decoder;

FIG. 4B is a set of results from a test using a two direction memory guided saccade task with pretraining of the BMI decoder;

FIG. 4C is a set of graphs showing the accuracy of the example BMI decoder with and without pretraining in the two direction memory guided saccade task;

FIG. 5A is a set of results from a test using an eight direction memory guided saccade task with no pretraining;

FIG. 5B is a set of results from a test using an eight direction memory guided saccade task with pretraining of the BMI decoder;

FIG. 5C is a set of graphs showing the accuracy of the example BMI decoder with and without pretraining in the eight direction memory guided saccade task;

FIG. 6 is a block diagram showing the process of a test involving a reach task;

FIG. 7A is a set of results from a test using a reach task with no pretraining of the BMI decoder;

FIG. 7B is a set of results from a test using the reach task with pretraining of the BMI decoder;

FIG. 7C is a set of graphs showing the accuracy of the example BMI decoder with and without pretraining of the BMI decoder in the reach task;

FIG. 8 is a set of images showing the process of aligning images from a pre-training session to produce an alignment image;

FIG. 9A are a set of confusion matrices for test subjects performing the eight direction memory guided saccade task over sessions conducted over a long period of time;

FIG. 9B are a set of graphs showing decoder stability for training and sessions for test subjects performing the eight direction memory guided saccade task over sessions conducted over a long period of time;

FIG. 9C is a set of graphs for the test subjects plotting mean angular error as a function of days between the training and testing session;

FIG. 10A shows images of vascular anatomy for test subjects over different test sessions over a relatively long time period;

FIG. 10B shows charts of pair-wise similarity between different vascular images for the test subjects;

FIG. 10C shows graphs of performance as a function of image similarity for the test subjects;

FIG. 11A shows a perspective view of an example cranial implant for fUSI imaging of the brain;

FIG. 11B shows a perspective view of the example cranial implant in FIG. 11A on the head of a test subject;

FIG. 11C is an isolated perspective view of the example cranial implant in FIG. 11A;

FIG. 11D is a graph showing spatial resolution versus coverage of various imaging techniques;

FIG. 11E is a diagram of the process of ultrafast acquisition of a series of fUSI images;

FIG. 12 is a perspective view of a blood flow phantom test system for testing the example cranial implant;

FIG. 13A is a series of power Doppler images from testing of the example implant in relation to the blood flow phantom system in FIG. 12;

FIG. 13B is a set of graphs showing power Doppler intensity and signal noise ratio attenuation from testing of the example implant in relation to the blood flow phantom system in FIG. 12;

FIG. 14A shows a series of in vivo ultrasound images of the coronal plane of a rat brain with different skull implant materials;

FIG. 14B shows a series of ultrasound images of the coronal plane and corresponding graphs of time course of the LGN region for the rat brain with different skull implant materials;

FIG. 14C is a series of graphs showing intensity, signal to noise ratio, and activation from the testing of different skull implant materials;

FIG. 15A are screen shots of ultrasound and power Doppler images of the brain of test participant prior to applying an example cranial implant;

FIG. 15B are screen shots of ultrasound images of the skull of the test participant with the example implant and a brain schematic;

FIG. 15C is a screen shot of a co-registration of the ultrasound imaging plane with an anatomical magnetic resonance image of the test participant;

FIG. 16A shows a testing environment for determining the suitability of the example cranial implant for ultrasound imaging of different tasks performed by the test participant;

FIG. 16B shows a timeline of a drawing task performed by the test participant used to determine the suitability of the example cranial implant for ultrasound imaging;

FIG. 16C shows a series of images that show the activation areas based on the drawing task test;

FIG. 16D shows a graph of means scaled ultrasound signals from regions of interest taken during the drawing task test;

FIG. 17A shows a power Doppler image of the vascular anatomy of the test participant during a guitar playing task;

FIG. 17B shows an activity map of the brain of the test participant during the guitar playing task; and

FIG. 17C shows a graph of means scaled ultrasound signals from regions of interest taken during the guitar playing task test.

### 7. DETAILED DESCRIPTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials specifically described.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

The present disclosure relates to a method and system for pretraining and/or stabilizing a neuroimaging-based brain-machine interface (BMI) using both new and previously collected brain state data such as image data from a brain during processing of commands. Pretraining based on the previously collected brain state input data from a brain during processing of commands reduces the need for calibrating the BMI and shortens the required training time. Stabilization helps maintain performance both within and across sessions, i.e., across time (hours, days, weeks, months, etc.). The example method and system also incorporates functional ultrasound neuroimaging. Thus, the example system assists in increasing service life of the BMI system, decreases invasiveness, and allows a wide range of data collection.

The example method consists of using image registration to align one, or multiple neuroimaging datasets to a common imaging field of view. In the pretraining scenario, the example method relies on co-registering neural populations (identified by imaging) and subsequently training a statistical model using the co-registered data from past sessions. For BMI stabilization, the example method regularly updates the co-registration to compensate for movement and/or changes in the recording field of view. This maintains and improves performance over time as additional data sets are incorporated into the training data set.

The example method also enables pretraining/stabilization between recording modalities. For example, a statistical model may be trained using data recorded by functional magnetic resonance imaging (fMRI). The resulting model may be used to decode intended behavioral signals from images such as those taken from functional ultrasound (fUS) neuroimaging data that is obtained from the brain of the subject.

The example BMI pre-training and stabilization method using live and pre-recorded 2D fUS neuroimaging data was successfully tested on a non-human primate performing various visual/motor behavioral tasks. The example method can be extended to novel BMI applications and across many modalities that produce neuro-functional images (such as 3D images, fUS images, MRI images, etc.) and non-image functional data, e.g., raw channel data.

Functional ultrasound (fUS) imaging is a recently developed technique that is poised to enable longer lasting, less invasive BMIs that can scale to sense activity from large regions of the brain. fUS neuroimaging is a means to collect brain state data that uses ultrafast pulse-echo imaging to sense changes in cerebral blood volume (CBV). fUS neuroimaging has a high sensitivity to slow blood flow (˜1 mm/s velocity) and balances good spatiotemporal resolution (100 μm; <1 sec) with a large and deep field of view (˜7 centimeters).

fUS neuroimaging possesses the sensitivity and field of view to decode movement intention on a single-trial basis simultaneously for two directions (left/right), two effectors (hand/eye), and task state (go/no-go). In this example, the fUS is incorporated into an online, closed-loop, functional ultrasound brain-machine interface (fUS-BMI). The example system allows decoding two or eight movement directions. The example decoder is stable across long periods of time after the initial training session.

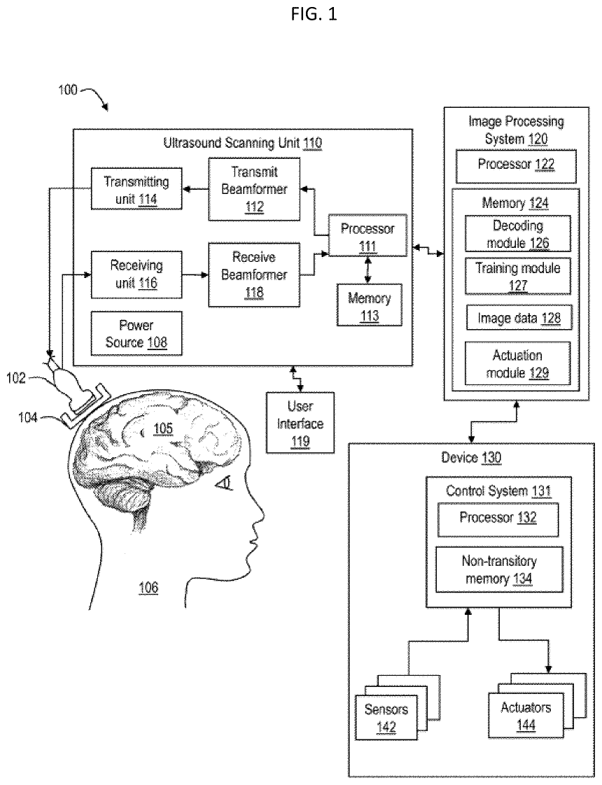

FIG. 1 illustrates a high-level block diagram of an example neural brain machine interface (BMI) system 100. The neural interface system 100 is configured to interface with the brain of a subject 106, perform, in real-time or near real-time, neuroimaging of the brain, and process, in real-time or near real-time, the neuroimaging data to determine one or more movement intentions of the subject 106. Further, the neural interface system 100 is configured to generate one or more control signals, in real time or near real-time, to a device 130 based on the one or more movement intentions determined from the neuroimaging data. The one or more movement intentions correspond to a cognitive state where a subject forms and develops motor planning activity before imagining, attempting, or executing a desired movement. As a non-limiting example, responsive to determining a movement intention of moving a desired effector (e.g., right arm) towards a desired direction (e.g., right), the neural interface system 100 may generate a control signal which may cause a corresponding prosthetic arm (e.g., right prosthetic arm) to move towards the desired direction (that is, right in this example) at the desired time. The desired effectors may be body effectors, including, but not limited to eyes, hands, arms, feet, legs, trunk, head, larynx, and tongue.

The neural interface system 100 comprises an ultrasound scanner or probe 102 for acquiring brain state data such as functional ultrasound (fUS) imaging of the brain, in real-time or near real-time. In particular, the ultrasound probe 102 may perform hemodynamic imaging of the brain to visualize changes in cerebral blood volume (CBV) using ultrafast Doppler angiography. fUS imaging enables a large field of view, and as such, a large area of the brain may be imaged using a single ultrasound probe. An example field of view of the ultrasound probe 102 may include various areas of posterior parietal cortex (PPC) of the brain including but not limited to lateral intraparietal (LIP) area, medial intraparietal (MIP) area, medial parietal area (MP), and ventral intraparietal (VIP) area.

Additionally or alternatively, fUS may be used to image hemodynamics in sensorimotor cortical areas and/or subcortical areas of the brain. For example, due to the large field of view of fUS systems, cortical areas deep within sulci and subcortical brain structures may be imaged in a minimally invasive manner that are otherwise inaccessible by electrodes. Further, fUS may be used to image hemodynamics in one or more of primary motor (M1), supplementary motor area (SMA), and premotor (PM) cortex of the brain.

In some examples, depending on a field of view of the ultrasound sound probe, larger or smaller areas of the brain may be imaged. Accordingly, in some examples more than one probe may be utilized for imaging various areas of the brain. However, in some examples, a single probe may be sufficient for imaging desired areas of the brain. As such, a number of probes utilized may vary and may be based at least on a desired imaging area, size of skull, and field of view of the probes. In this way, by using fUS, neural activity can be visualized not only larger areas of brain but also in deeper areas of brain with improved spatial and temporal resolution and sensitivity.

In one example, the probe 102 may be positioned within a chamber 104 coupled to the subject's skull. For example, a cranial window may be surgically opened while maintaining a dura underneath the cranium intact. The probe 102 and the chamber 104 may be positioned over the cranial window to enable neuroimaging via the cranial window. In some examples, an acoustic coupling gel may be utilized to place the probe 102 in contact with the dura mater above the brain 105 within the chamber 104.

In another example, a neuroplastic cranial implant that replaces a portion of a subject's skull may be used. The neuroplastic cranial implant may comprise one or more miniaturized probes, for example. Implanted probes may also perform data processing and/or decoding in addition to transmitting data and/or power wirelessly through the scalp to a receiver.

In another example, a sonolucent material is used to replace a portion of a subject's skull (cranioplasty) above a brain region of interest to facilitate fUS imaging as will be explained below. One or more ultrasound probes can be positioned afterward above the scalp, implant, and brain region of interest in a non-invasive way, for example, via a cranial cap comprising a stereotaxic frame supporting one or more ultrasound probes. In yet another example, the one or more probes may be positioned above the scalp and skull without a craniotomy, for example, via a cranial cap comprising a stereotaxic frame supporting one or more ultrasound probes.

Further, the ultrasound probe 102 and its associated skull coupling portions (e.g., chamber 104, neuroplastic implants, stereotaxic frames, etc.) may be adapted for various skull shapes and sizes (e.g., adults, infants, etc.). Furthermore, the ultrasound probe 102 and the associated skull coupling portions may enable imaging of the brain while the subject is awake and/or moving. Further, in one example, the probe 102 may be placed surface normal to the brain on top of the skull in order to acquire images from the posterior parietal cortex of the brain for movement decoding. However, in order to image a larger area of the brain or multiple brain areas, additional probes, each positioned at any desired angle with respect to the brain may be utilized.

The neural interface system 100 further includes an ultrasound scanning unit 110 (hereinafter “scanning unit 110” or “scanner 110”) communicatively coupled to the ultrasound probe 102, and a real-time signal analysis and decoding system 120 communicatively coupled to the ultrasound scanning unit 110. Communication between the probe 102 and the scanning unit 110 may be wired, or wireless, or a combination thereof. Similarly, communication between the scanning unit 110 and the real-time signal analysis and decoding system 120 may be wired, or wireless, or a combination thereof. While the present example shows the scanning unit 110 and the real-time signal analysis and decoding system 120 separately, in some examples, the scanning unit 110 and the real-time signal analysis and decoding system 120 may be configured as a single unit. Thus, the ultrasound images acquired via the probe 102 may be processed by an integrated/embedded processor of the ultrasound scanner 110. In some examples, the real-time signal analysis and decoding system 120 and the scanning unit 110 may be separate but located within a common room. In some examples, the real-time signal analysis and decoding system 120 may be located in a remote location from the scanning unit 110. For example, the real-time signal analysis and decoding system may operate in a cloud-based server that has a distinct and remote location with respect to other components of the system 100, such as the probe 102 and scanning unit 110. Optionally, the scanning unit 110 and the real-time signal analysis and decoding system 120 may be a unitary system that is capable of being moved (e.g., portably) from room to room. For example, the unitary system may include wheels or be transported on a cart. Further, in some examples, the probe 102 may include an integrated scanning unit and/or an integrated real-time signal analysis and decoding system, and as such, fUS signal processing and decoding may be performed via the probe 102, and the decoded signals may be transmitted (e.g., wirelessly and/or wired) directly to the device 130.

In this example, the neural interface system 100 includes a transmit beamformer 112 and transmitting unit 114 that drives an array of transducer elements (not shown) of the probe 102. The transducer elements may comprise piezoelectric crystals (or semiconductor based transducer elements) within probe 102 to emit pulsed ultrasonic signals into the brain 105 of the subject. In one example, the probe 102 may be a linear array probe, and may include a linear array of a number of transducer elements. The number of transducer elements may be 128, 256, or another number suitable for ultrasound imaging of the brain. Further, in some examples, the probe may be a phased array probe. Furthermore, any type of probe that may be configured to generate plane waves may be used.

Ultrasonic pulses emitted by the transducer elements are back-scattered from structures in the body, for example, blood vessels and surrounding tissue, to produce echoes that return to the transducer elements. In one example, a conventional ultrasound imaging with focused beam may be performed. The echoes are received by a receiving unit 116. The received echoes are provided to a receive beamformer 118 that performs beamforming and outputs an RF signal. The RF signal is then provided to the processor 111 that processes the RF signal. Alternatively, the processor 111 may include a complex demodulator (not shown) that demodulates the RF signal to form IQ data pairs representative of the echo signals. In some examples, the RF or IQ signal data may then be provided directly to a memory 114 for storage (for example, temporary storage).

In order to detect CBV changes in the brain, Doppler ultrasound imaging may be performed. Doppler ultrasound imaging detects movement of red blood cells by repeating ultrasonic pulses and evaluating temporal variations of successive backscattered signals. In one embodiment, ultrafast ultrasound imaging may be utilized based on plane wave emission for imaging CBV changes in brain tissue. Plane wave emission involves simultaneously exciting all transducer elements of the probe 102 to generate a plane wave. Accordingly, the ultrafast ultrasound imaging includes emitting a set of plane waves at titled angles in a desired range from a start degree to a final degree tilt of the probe 102 at a desired angular increment (e.g., 1 degree, 2 degrees, 3 degrees, etc.). An example desired range may be from −15 degrees to 15 degrees. In some examples, the desired range may be from approximately −30 degrees to +30 degrees. The above examples of ranges are for illustration, and any desired range may be implemented based on one or more of area, depth, and imaging system configurations. In some examples, an expected cerebral blood flow velocity may be considered in determining the desired range for imaging.

In one non-limiting example, the set of plane waves may be emitted at tilted angles of −6 to 6° at 3 degree increments. In another non-limiting example, the set of plane waves may be emitted at tilted angles from −7° to 8° at 1-degree increments.

Further, in some examples, a 3-dimensional (3D) fUS sequence may be utilized for imaging one or more desired areas of the brain. In one example, in order to acquire 3D fUS sequences, a 4-axis motorized stage including at least one translation along the x, y, and/or z axes, and one rotation about the z axis may be utilized. For example, a plurality of linear scans may be performed while moving the probe to successive planes to perform a fUS acquisition at each position to generate 3D imaging data. In another example, in order to acquire 3D fUS sequences, a 2D matrix array or row-column array probe may be utilized to acquire 3D imaging data in a synchronous manner, i.e., without moving the probe. 3D imaging data thus obtained may be processed for evaluating hemodynamic activity in the targeted areas of the brain and decoding movement intentions may be decoded. Thus, the systems and methods described herein for movement intention decoding using fUS may also be implemented by using 3D fUS imaging data without departing from the scope of the disclosure.

Imaging data from each angle is collected via the receiving unit 116. The backscattered signals from every point of the imaging plane are collected and provided to a receive beamformer 118 that performs a parallel beamforming procedure to output a corresponding RF signal. The RF signal may then be utilized by the processor 111 to generate corresponding ultrasonic image frames for each plane wave emission. Thus, a plurality of ultrasonic images may be obtained from the set of plane wave emissions. A total number of the plurality of ultrasonic images is based on acquisition time, a total number of angles, and pulse repetition frequency.

The plurality of ultrasonic images obtained from the set of plane wave emissions may then be added coherently to generate a high-contrast compound image. In one example, coherent compounding includes performing a virtual synthetic refocusing by combining the backscattered echoes of the set of plane wave emissions. Alternatively, the complex demodulator (not shown) may demodulate the RF signal to form IQ data representative of the echo signals. A set of IQ demodulated images may be obtained from the IQ data. The set of IQ demodulated may then be coherently summed to generate the high-contrast compound image. In some examples, the RF or IQ signal data may then be provided to the memory 113 for storage (for example, temporary storage).

Further, in order to image brain areas with desired spatial resolution, the probe 102 may be configured to transmit high-frequency ultrasonic emissions. For example, the ultrasound probe may have a central frequency of at least 5 MHz for fUS imaging for single-trial decoding. Functional hyperemia (that is, changes in cerebral blood flow or volume corresponding to cognitive function) arises predominantly in microvasculature (sub-millimeter), and as such high-frequency ultrasonic emissions are utilized to improve spatial resolution to detect such signals. Further, as fUS enables brain tissue imaging at greater depths, movement intention decoding can be efficiently accomplished without invasive surgery that may be needed for an electrophysiology based BMI.

The processor 111 is configured to control operation of the neural interface system 100. For example, the processor 111 may include an image-processing module that receives image data (e.g., ultrasound signals in the form of RF signal data or IQ data pairs) and processes image data. For example, the image-processing module may process the ultrasound signals to generate volumes or frames of ultrasound information (e.g., ultrasound images) for display to the operator. In system 100, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D). When multiple two-dimensional (2D) images are obtained, the image-processing module may also be configured to stabilize or register the images.

Further, acquired ultrasound information may be processed in real-time or near real-time during an imaging session (or scanning session) as the echo signals are received. In some examples, an image memory may be included for storing processed slices of acquired ultrasound information that may be accessed at a later time. The image memory may comprise any known data storage medium, for example, a permanent storage medium, removable storage medium, and the like. Additionally, the image memory may be a non-transitory storage medium.

In operation, an ultrasound system may acquire data, for example, volumetric data sets by various techniques (for example, 3D scanning, real-time 3D imaging, volume scanning, 2D scanning with probes having positioning sensors, scanning using 2D or matrix array probes, and the like). In some examples, the ultrasound images of the neural interface system 100 may be generated, via the processor 111, from the acquired data, and displayed to an operator or user a display device of a user interface 119 communicatively coupled to the scanning unit 110.

In some examples, the processor 111 is operably connected to the user interface 119 that enables an operator to control at least some of the operations of the system 100. The user interface 119 may include hardware, firmware, software, or a combination thereof that enables a user (e.g., an operator) to directly or indirectly control operation of the system 100 and the various components thereof. The user interface 119 may include a display device (not shown) having a display area (not shown). In some embodiments, the user interface 119 may also include one or more input devices (not shown), such as a physical keyboard, mouse, and/or touchpad. In an exemplary embodiment, the display device 118 is a touch-sensitive display (e.g., touchscreen) that can detect a presence of a touch from the operator on the display area and can also identify a location of the touch in the display area. The display device also communicates information from the processor 111 to the operator by displaying the information to the operator. The display device 118 may be configured to present information to the operator during one or more of an imaging session, and training session. The information presented may include ultrasound images, graphical elements, and user-selectable elements, for example.

The neural interface system 100 further includes the real-time signal analysis and decoding system 120 which may be utilized for decoding neural activity in real-time. In one example, neural activity may be determined based on hemodynamic changes, which can be visualized via fUS imaging. As discussed above, while the real-time signal analysis and decoding system 120 and the scanning unit 110 are shown separately, in some embodiments, the real-time signal analysis and decoding system 120 may be integrated within the scanning unit and/or the operations of the real-time signal analysis and decoding system 120 may be performed by the processor 111 and memory 113 of the scanning unit 110.

The real-time signal analysis and decoding system 120 is communicatively coupled to the ultrasound scanning unit 110, and receives ultrasound data from the scanning unit 110. In one example, the real-time signal analysis and decoding system 120 receives compounded ultrasound images, in real-time or near real-time, generated via the processor 111 based on plane wave emission via probe 102. The real-time signal analysis and decoding system 120 includes non-transitory memory 113 that stores a decoding module 126. The decoding module 126 may include a decoding model that is trained for decoding movement intentions of a subject by correlating neural activity in the brain using the compounded ultrasound images received from the scanning unit 110 with movement intention. The decoding model may be a machine learning model that is pre-trained with data from previous sessions with the subject as will be explained herein. Accordingly, the decoding module 126 may include instructions for receiving imaging data acquired via an ultrasound probe, and implementing the decoding model for determining onc or more movement intentions of a subject. In one example, the imaging data may include a plurality of CBV images generated by a performing power Doppler imaging sequence via an ultrasound probe 102. In one example, the CBV images are compound ultrasound images generated based on Doppler imaging of the brain.

Non-transitory memory 124 may further store a training module 127, which includes instructions for training the machine learning model stored in the decoding module 108. Training module 127 may include instructions that, when executed by processor 122, cause real-time signal analysis and decoding system 120 to train the decoding model that has been pre-trained using a training dataset that may include imaging datasets from previous training sessions as will be described below. Example protocols implemented by the training module 110 may include learning techniques such as gradient descent algorithm, such that the decoding model can be trained and can classify input data that were not used for training.

Non-transitory memory 124 may also store an inference module (not depicted) that comprises instructions for testing new data with the trained decoding model. Further, non-transitory memory 124 may store image data 128 received from the ultrasound scanning unit 110. In some examples, the image data 128 may include a plurality of training datasets generated via the ultrasound scanning unit 110.

Real-time signal analysis and decoding system 120 may further include a user interface (not shown). The user interface may be a user input device, and may comprise one or more of a touchscreen, a keyboard, a mouse, a trackpad, a motion sensing camera, an eye tracking camera, and other device configured to enable a user to interact with and manipulate data within the processing system 120.

The real-time signal analysis and decoding system 120 may further include an actuation module 129 for generating one or more actuation signals in real-time based on one or more decoded movement intention (e.g., determined via the decoding model). In one example, the actuation module 129 may use a derived transformation rule to map an intended movement signal, s, into an action, a, for example, a target. Statistical decision theory may be used to derive the transformation rule. Factors in the derivations may include the set of possible intended movement signals, S, and the set of possible actions, A. The neuro-motor transform, d, is a mapping for S to A. Other factors in the derivation may include an intended target θ and a loss function which represents the error associated with taking an action, a, when the true intention was θ. These variables may be stored in a memory device, e.g., memory 124.

In some examples, two approaches may be used to derive the transformation rule: a probabilistic approach, involving the intermediate step of evaluating a probabilistic relation between s and θ and subsequent minimization of an expected loss to obtain a neuro-motor transformation (i.e., in those embodiments of the invention that relate to intended movement rather than, e.g., emotion); and a direct approach, involving direct construction of a neuro-motor transformation and minimizing the empirical loss evaluated over the training set. Once the actuation module maps an intended movement signal to an action, the actuation module 129 may generate an actuation signal indicative of the cognitive signal (that is, intended movement signal) and transmit the actuation signal to a device control system 131 of a device 130. The device control system 131 may use the actuation signal to adjust operation of one or more actuators 144, that may be configured to execute a movement based on the actuation signals generated by the actuation module 129. For example, adjusting the operation of one or more actuators 144 may include mimicking the subject's intended movement or perform another task (e.g., move a cursor, turn off the lights, perform home environmental temperature control adjustments) associated with the cognitive signal.

Thus, based on decoded intended movements, via the decoding module 126, one or more actuation signals may be transmitted to the device 130 communicatively coupled to the neural interface system 100. Further, the control system 131 is configured to receive signals from and send signals to the real-time signal analysis and decoding system 120 via a network. The network may be wired, wireless, or various combinations of wired and wireless. In some examples, the actuation module 129 may be configured as a part of the device 130. Accordingly, in some examples, the device 130 may generate one or more actuation signals based on movement intention signals generated by an integrated decoding module. As a non-limiting example, based on a movement intention (e.g., move right hand to the right), the actuation module 129 may generate, in real-time, an actuation signal which is transmitted, in real-time, to the control system 131. The actuation signal may then be processed by the device control system 131 and transmitted to a corresponding actuator (e.g., a motor actuator of a right hand prosthetic limb) causing the actuator to execute the intended movement.

The device 130 may be, for example, a robotic prosthetic, a robotic orthotic, a computing device, a speech prosthetic or speller device, or a functional electrical stimulation device implanted into the subject's muscles for direct stimulation and control or any assistive device. In some examples, the device 130 may be a smart home device, and the actuation signal may be transmitted to the smart home controller to adjust operation of the smart home device (e.g., a smart home thermostat, a smart home light, etc.) without the need for using a prosthetic limb. Thus, the neural interface system 100 may interface with a control system of a device, without the use of a prosthetic limb. In some examples, the device may be a vehicle and the actuation signal may be transmitted to a vehicle controller to adjust operation of the vehicle (e.g., to lock/unlock door, to open/close door, etc.). Indeed, there are a wide range of tasks that can be controlled by a prosthetic that receives instruction based on the cognitive signals harnessed in various embodiments of the present disclosure. Reaches with a prosthetic limb could be readily accomplished. An object such as a cursor may be moved on a screen to control a computer device. Alternatively, the mental/emotional state of a subject (e.g., for paralyzed patients) may be assessed, as can intended value (e.g., thinking about a pencil to cause a computer program (e.g., Visio) to switch to a pencil tool, etc.). Other external devices that may be instructed with such signals, in accordance with alternate embodiments of the present disclosure, include, without limitation, a wheelchair or vehicle; a controller, such as a touch pad, keyboard, or combinations of the same; and a robotic hand.

In some examples, the neural interface system 100 may be communicatively coupled one or more devices. Accordingly, the neural interface system 100 may transmit control signals (based on decoded intention signals) simultaneously or sequentially to more than one device communicatively coupled to the neural interface system 100. For example, responsive to decoding movement intentions, the real-time signal analysis and decoding system 120 may generate and transmit a first control signal (e.g., based on decoding a first intended effector, such as arms, first direction, and/or first action) to a first device (e.g. robotic limb to grasp a cup) and simultaneously or sequentially, generate and transmit a second control signal (e.g., based on a second decoded intended effector such as eyes, second direction, and/or second action) to a second device (e.g., computer for cursor movement). Thus, in some examples, the neural interface system 100 may be configured to communicate with and/or adjust operation of more than one device.

The actuation module 129 may use a feedback controller to monitor the response of the device, via one or more sensors 142, and compare it to, e.g., a predicted intended movement, and adjust actuation signals accordingly. For example, the feedback controller may include a training program to update a loss function variable used by the actuation module 129.

The subject may be required to perform multiple trials to build a database for the desired hemodynamic signals corresponding to a particular task. As the subject performs a trial, e.g., a reach task or brain control task, the neural data may be added to a database for pre-training of the decoder for a current session. The memory data may be decoded, e.g., using a trained decoding model, and used to control the prosthetic to perform a task corresponding to the cognitive signal. Other predictive models may alternatively be used to predict the intended movement or other cognitive instruction encoded by the neural signals.

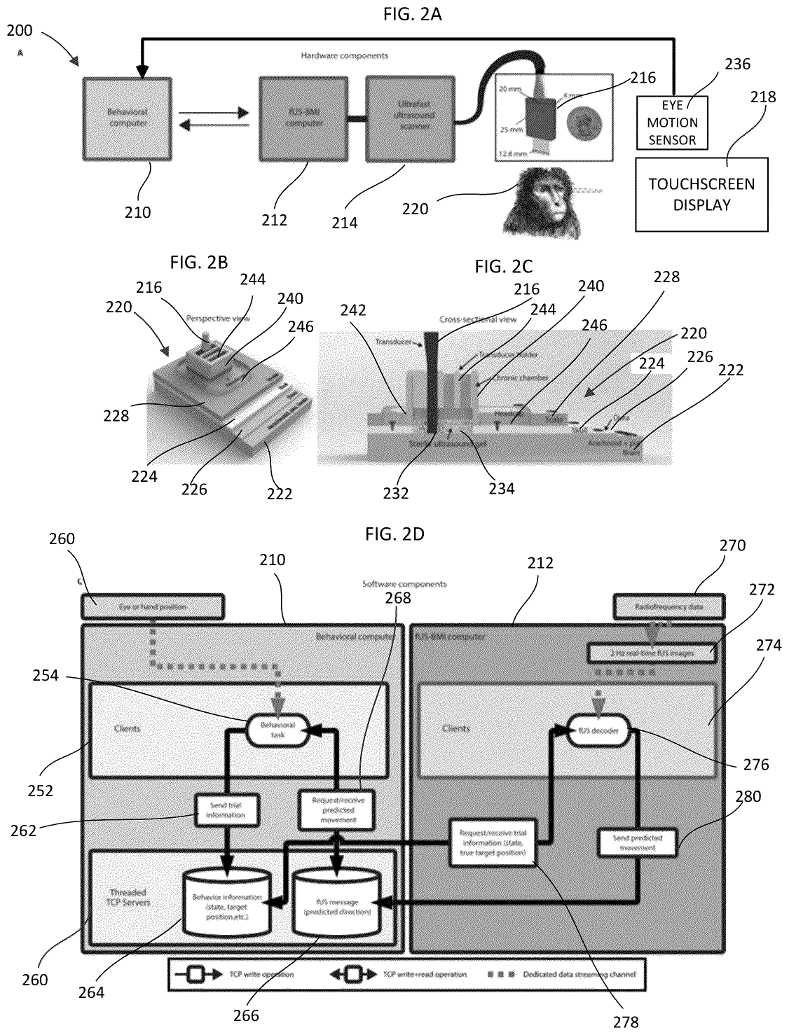

FIG. 2A shows a hardware block diagram of a test system 200 that was used to establish the effectiveness of the example BMI pre-training method. The test system 200 includes a behavioral computer system 210, a functional ultrasound (fUS) BMI computer 212, an ultra-fast ultrasound scanner 214, and an ultrasonic transducer 216. In the example test system 200, the transducer 216 is a 128-element miniaturized linear array probe, 15.6 MHz center frequency, 0.1 mm pitch available from Vermon of Tours, France that is paired with a real-time ultrafast ultrasound acquisition system of the scanner 214 to stream 2 Hz fUS images. Testing was performed using the system on two nonhuman primates (NHPs) as they performed memory-guided eye movements.

FIG. 2B shows a perspective view of the ultrasonic transducer 216 attached to the head of a test subject 220. FIG. 2C a cross section of the ultrasonic transducer 216 attached to the head of the test subject 220. The test subject 220 has a brain 222 protected by a skull 224. A dura layer 226 insulates the skull 224 from the brain 222. The skull 224 is covered by a scalp 228.

The transducer 216 includes a transducing surface 232 that is inserted in a sterile ultrasound gel layer 234 applied to the brain 222. A rectangular chronic chamber 240 is inserted through an aperture 242 created in the skull 224. The rectangular chronic chamber 240 is attached to a transducer holder 244 that holds the transducer 216. The chronic chamber 240 is attached to a headcap 246 that is inserted over the aperture 242 on the scalp 228. Thus, the chronic chamber 240 and holder 244 hold the transducer 216 such that the sensing end 232 is in contact with the brain 222.

In the tests, the transducer surface 232 was positioned normal to the brain 222 above the dura mater 226 and recorded from coronal planes of the left posterior parietal cortex (PPC), a sensorimotor association area that uses multisensory information to guide movements and attention. This technique achieved a large field of view (12.8 mm width, 16 mm depth, 400 μm plane thickness) while maintaining high spatial resolution (100 μm×100 μm in-plane). This allowed streaming high-resolution hemodynamic changes across multiple PPC regions simultaneously, including the lateral (LIP) and medial (MIP) intraparietal cortex. Previous research has shown that the LIP and MIP are involved in planning eye and reach movements respectively. This makes the PPC a good region from which to record effector-specific movement signals. Thus, the sensing end 232 of the transducer 216 was positioned above the dura 226 with the ultrasound gel layer 234. The ultrasound transducer 216 was positioned in the recording sessions using the slotted chamber plug holder 244. The imaging field of view was 12.8 mm (width) by 13-20 mm (height) and allowed the simultaneous imaging of multiple cortical regions of the brain 222, including the lateral intraparietal area (LIP), the medial intraparietal area (MIP), the ventral intraparietal area (VIP), Area 7, and Area 5.

The tests employed Neuroscan software interfaced with MATLAB 2019b for the real-time fUS-BMI and MATLAB 2021a for all other analyses in the fUS-BMI computer 212. For each NHP test subject, a cranial implant containing a titanium head post was placed over the dorsal surface of the skull and a craniotomy positioned over the posterior parietal cortex of the brain 222. The dura 226 underneath the craniotomy was left intact. The craniotomy was covered by a 24×24 mm (inner dimension) chamber 240. For each recording session, the custom 3D-printed polyetherimide slotted chamber plug 240 that held the ultrasound transducer 216 was used. This transducer 216 allowed the same anatomical planes to be consistently acquired on different days.

As shown in FIG. 2A, the test subject 220 is positioned relative to a touchscreen display 218 that displays visual cues. In the behavioral setup, the NHP test subjects sat in a primate chair facing the monitor or touchscreen 218. The touchscreen monitor 218 was positioned approximately 30 cm in front of the NHP. The touchscreen was positioned on each day so that the NHP could reach all the targets on the display screen with his fingers but could not rest his palm on the screen. In this example, the display screen is an ELO IntelliTouch touchscreen display available from ELO Touch Solutions, Inc. of Milpitas, California.

An eye motion sensor 236 followed the eyes of the test subject 220 and generates eye position data that is sent to the behavioral computer system 210. Eye position is tracked at 500 Hz using the eye motion sensor 236, which was an infrared eye tracker such as an EyeLink 1000 available from SR Research Ltd. of Ottawa, Canada). Touch is tracked using the touchscreen display screen 218. Visual stimuli such as the cues were presented using custom Python 2.7 software based on PsychoPy. Eye and hand position was recorded simultaneously with the stimulus and timing information and stored for offline analysis.

The programmable high-framerate ultrasound scanner 214 is a Vantage 256 scanner available from Verasonics of Kirkland, WA. The scanner 214 was used to drive the ultrasound transducer 216 and collect pulse echo radio frequency data. Different plane-wave imaging sequences were used for real-time and anatomical fUS neuroimaging.

The real-time fUS neuroimaging performed by the computer 212 in this example is a custom-built computer running NeuroScan Live software available from ART INSERM U1273 & Iconcus of Paris, France attached to the 256-channel Verasonics Vantage ultrasound scanner 214. This software implemented a custom plane-wave imaging sequence optimized to run in real-time at 2 Hz with minimal latency between ultrasound pulses and power Doppler image formation. The sequence used a pulse-repetition frequency of 5500 Hz and transmitted plane waves at 11 tilted angles. The tilted plane waves were compounded at 500 Hz. Power Doppler images were formed from 200 compounded B-mode images (400 ms). To form the power Doppler images, the software used an ultrafast power Doppler sequence with an SVD clutter filter that discarded the first 30% of components. The resulting power Doppler images were transferred to a MATLAB instance in real-time and used by the fUS-BMI computer 212. The prototype 2 Hz real-time fUSI system had a 0.71±0.2 second (mean±STD) latency from ultrasound pulse to image formation. Each fUSI image and associated timing information were saved for post hoc analyses.

For anatomical fUS neuroimaging a custom plane-wave imaging sequence was used to acquire an anatomical image of the vasculature. A pulse repetition frequency of 7500 Hz and transmitted plane waves at 5 angles ([−6°,−3°, 0°, 3°, 6°]) with 3 accumulations was used. The 5 angles were coherently compounded from 3 accumulations (15 images) to create one high-contrast ultrasound image. Each high-contrast image was formed in 2 ms, i.e., at a 500 Hz framerate. A power Doppler image of the brain of the NHP test subjects was formed using 250 compounded B-mode images collected over 500 ms. Singular value decomposition was used to implement a tissue clutter filter and separate blood cell motion from tissue motion.

FIG. 2D shows a block diagram of the software components of the test system 200. The software components are executed by the behavioral computer system 210 and the fUS BMI computer 212. A set of position inputs 250 that include either eye or hand position are fed into the behavioral computer system 210. In this example, the behavioral computer system 210 includes a client application 252 that is associated with a particular behavioral task 254. In this example, the behavioral task 254 may include tracking movement in two directions or eight directions, a reach task, or other tasks as explained herein. The behavioral computer system 210 also includes TCP threaded database servers 260. The threaded TCP server 260 was designed in Python 2.7 to receive, parse, and send information between the computer running the PsychoPy behavior software and the real-time fUS-BMI computer 212. Upon queries from the fUS-BMI computer 212, the server 260 transferred task information, including task timing and actual movement direction, to the real-time ultrasound system. The real-time ultrasound system includes the transducer 216 and the scanner 214. The client-server architecture is specifically designed to prevent data leaks, i.e., the actual movement direction is never transmitted to the fUS-BMI computer 212 until after a successful trial had ended. The average server write-read-parse time was 31+/−1 (mean±STD) ms during offline testing between the two desktop Windows computers 210 and 212 on a local area network (LAN).

Trial information data 262 was sent to a behavior information database 264 by the behavioral task 254. Previous predictions are stored in an ultrasonic image database 266. The behavioral task 254 requests and receives predicted movement data 268 from the database 266 to perform the behavioral task 254.

Radio frequency data 270 from the transducer 216 is input into the computer 212. The computer 212 breaks down the radio frequency data 270 into real-time functional ultrasound images 272. The computer 212 executes client applications 274 that includes a BMI decoder 276 that that converts the fUSI images into computer commands. The BMI decoder 276 requests and receives trial information 278 such as state and true target information from the behavior information database 264. After receiving the trial information, the BMI decoder 276 sends a predicted movement 280 to the fUS message database 266. Thus, the TCP server 260 also received the fUS-BMI prediction 280 and passed the prediction to the PsychoPy software of the decoder 276 when queried.

The real-time 2 Hz fUS images 272 are streamed into the BMI decoder 276 that uses principal component (PCA) and linear discriminant analysis (LDA) to predict planned movement directions. The BMI output is used to directly control the behavioral task of the respective tests.

There were three steps to decoding movement intention in real-time for the brain machine interface executed by the BMI computer 212 of the system 200: 1) applying preprocessing to a rolling data buffer; 2) training the classifier in the decoder 276; and 3) decoding movement intention in real-time using the trained classifier. The time for preprocessing, training, and decoding is dependent upon several factors, including the number of trials in the training set, CPU load from other applications, the field of view, and the classifier algorithm (PCA+LDA vs cPCA+LDA). In the worst cases during offline testing, the preprocessing, training, and decoder respectively took approximately 10, 500 ms, and 60 ms.

Before feeding the power Doppler images into the classification algorithm, two preprocessing operations were applied to a rolling 60-frame (30 seconds) buffer. First, a rolling voxel-wise z-score was performed over the previous 60 frames (30 seconds). Second, a pillbox spatial filter was applied with a radius of 2 pixels to each of the 60 frames in the buffer.

The fUS-BMI computer 212 makes a prediction at the end of the memory period using the preceding 1.5 seconds of data (3 frames) and passes this prediction to the behavioral control system 254 via the threaded TCP-based server 260 in FIG. 2D. Different classification algorithms for fUS-BMI were used in the 2-direction and 8-direction tasks. For decoding two directions of eye or hand movements, a class wise principal component analysis (cPCA) and linear discriminant analysis (LDA) was used. This is a method well suited to classification problems with high dimensional features and low numbers of samples. This method is identical to that used previously for offline decoding of movement intention but has been optimized for online training and decoding. Briefly, the cPCA was used to dimensionally reduce the data while keeping 95% variance of the data. LDA was then used to improve the class separability of the cPCA-transformed data.

For decoding eight directions of eye movements, a multi-coder approach was used where the horizontal (left, center, or right) and vertical components (down, center, or up) were separately predicted and combined to form the final prediction. As a result of this separate decoding of horizontal and vertical movement components, “center” predictions are possible (horizontal-center and vertical-center) although this is not one of the eight peripheral target locations. To perform the predictions, principal component analysis (PCA) and LDA was used. The PCA was used to reduce the dimensionality of the data while keeping 95% of the variance in the data. The LDA was used to predict the most likely direction. The PCA+LDA method was selected over the cPCA+LDA for 8-direction decoding because in pilot offline data, the PCA+LDA multi-coder worked marginally better than the cPCA+LDA method for decoding eight movement directions with a limited number of training trials.

The tests were conducted on two healthy 14-year-old male rhesus macaque monkeys (Macaca mulatta) weighing 14-17 kg designated as NHP test subjects L and P. The test monkeys were implanted with the ultrasound transducers such as the transducer 216 in FIGS. 2B-2C. All surgical and animal care procedures were approved by the California Institute of Technology Institutional Animal Care and Use Committee and complied with the Public Health Service Policy on the Humane Care and Use of Laboratory Animals.

The NHP test subjects performed several different memory-guided movement tasks including the memory guided saccade tasks and the memory-guided reach task. In the memory-guided saccade tasks, the NHP test subjects fixated on a center cue for 5±1 seconds. A peripheral cue appeared for 400 ms in a peripheral location (either chosen from 2 or 8 possible target locations) at 20° eccentricity. The NHP subject kept fixation on the center cue through a memory period (5±1 s) where the peripheral cue was not visible. The NHP test subject then executed a saccade to the remembered location once the fixation cue was extinguished. If the eye position of the NHP test subject was within a 7° radius of the peripheral target, the target was re-illuminated and stayed on for the duration of the hold period (1.5±0.5 s). The NHP test subject received a liquid reward of 1000 ms (0.75 mL) for successful task completion. There was an 8±2 second intertrial interval before the next trial began. Fixation, memory, and hold periods were subject to timing jitter sampled from a uniform distribution to prevent the NHP test subject from anticipating task state changes.

The memory-guided reach task was similar to the memory guided saccade tasks, but instead of fixation, the NHP used fingers on a touchscreen. Due to space constraints, eye tracking was not used concurrently with the touchscreen, i.e., only hand or eye position was tracked, not both.

For the memory-guided BMI task, the NHP test subject performed the same fixation steps using his eye or hand position, but the movement phase was controlled by the fUS-BMI test system 200. Critically, the NHP was trained to not make an eye or hand movements from the center cue until at least the reward was delivered. For this task variant, the NHP received a liquid reward that was 1000 ms (0.75 mL) for successfully maintaining fixation/touch for correct fUS-BMI predictions and 100 ms (0.03 mL) for successfully maintaining fixation/touch for incorrect fUS-BMI predictions. This was done to maintain NHP motivation even if the fUS-BMI was inaccurate.

The fUS-BMI decoder was retrained during the inter-trial interval whenever the training set changed. For the real-time experiments, each successful trial was automatically added to the training set. In the training phase, successful trials were defined as the NHP test subject performing the movement to the correct target and receiving his juice reward. In the BMI mode, successful trials were defined as a correct prediction plus the NHP test subject maintaining fixation until juice delivery.

For experiments using data from a previous session, the model for the BMI decoder 276 was trained using all the valid trials in the training data set 374 from the previous session upon initialization of the fUSI-BMI. A valid trial was defined as any trial that reached the prediction phase, regardless of whether the correct class was predicted. The BMI decoder 276 was then retrained after each successful trial was added to the training set 370 during the current session.

For post hoc experiments analyzing the effect of using only data from a given session, all trials where the test subject monkey received a reward were considered as successful and retrained after each trial. This was done to keep the many training trials where the NHP test subject maintained fixation throughout the trial despite an incorrect BMI prediction.

At the beginning of each experimental session, a single anatomical fUS image was acquired from the transducer 216 using the beam forming process showing the macro- and mesovasculature within the imaging field of view. For sessions where previous data was used as the initial training set for the fUS-BMI, a semi-automated intensity-based rigid-body registration was performed between the new anatomical image and the anatomical image acquired in a previous session. The MATLAB “imregtform” function was used with the mean square error metric and a regular step gradient descent optimizer to generate an initial automated alignment of the previous anatomical image to the new anatomical image. If the automated alignment had misaligned the two images, the anatomical image from the previous session was manually shifted and rotated using a custom MATLAB GUI. The final rigid body was applied to transform the training data from the previous session, thus aligning the previous session to the new session.

Summary statistics were reported as XX±XX are mean±SEM.

The recorded real-time fUS images were used to simulate the effects of different parameters on fUS-BMI performance, such as using only current session data without pretraining. To do this, the recorded fUS images and behavioral data were fed frame by frame through the same fUS-BMI function used for the closed-loop, online fUS-BMI. To dynamically build the training set 374, all trials reaching the end of the memory phase were added to the training set 374 regardless of whether the offline fUS-BMI predicted the correct movement direction. This was done because the high possible error rate from bad predictions meant that building the training set from only correctly predicted trials could be imbalanced for the different directions and possibly contain insufficient trials to train the model, e.g., no correct predictions for certain directions would prevent the model from being able to predict that direction.

A circular region of interest (ROI; 200 μm radius) was defined and moved the circular region of interest sequentially across all voxels in the imaging field of view. For each ROI, offline decoding with 10-fold cross-validation was performed using either the cPCA+LDA (2-directions) or PCA+LDA (8-directions) algorithm. The voxels fully contained with each ROI were used in both algorithms. The mean performance across the cross-validation folds was assigned to the center voxel of the ROI. To visualize the results, the performance (mean absolute angular error or accuracy) of the 10% most significant voxels was overlaid on the anatomical vascular map from the session.

In the tests, the two NHP test subjects initially performed instructed eye movements to a randomized set of two or eight peripheral targets to build the initial training set for the decoder 276. The fUS activity during the delay period preceding successful eye movements were used to train the decoder 276. After 100 successful training trials, the system 200 was switched to a closed-loop BMI mode where the movement directions came from the fUS-BMI computer 212. During the closed-loop BMI mode, the NHP test subject continued to fixate on the center cue until after the delivery of the liquid reward. During the interval between a successful trial and the subsequent trial, the decoder 276 was rapidly retrained, continuously updating the decoder model as each NHP test subject used the fUS-BMI system.

A first set of tests was directed toward online decoding of two eye movement directions. To demonstrate the feasibility of an example fUS-BMI system, online, closed-loop decoding of two movement directions was performed. A second set of tests was directed toward online decoding of eight eye movement directions.

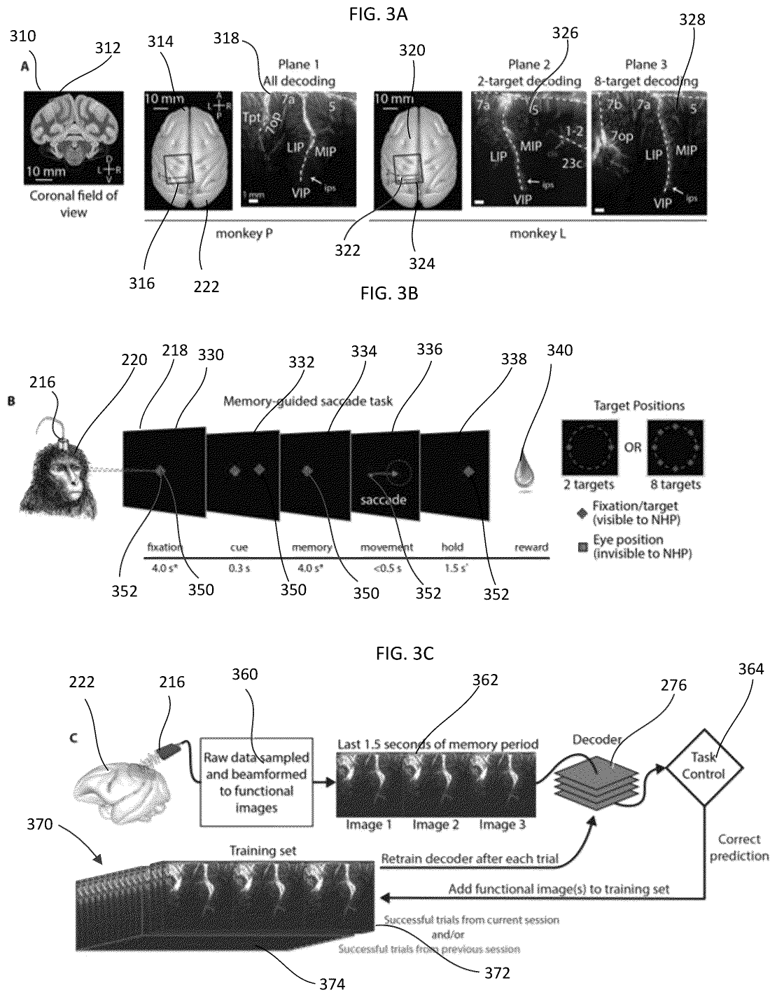

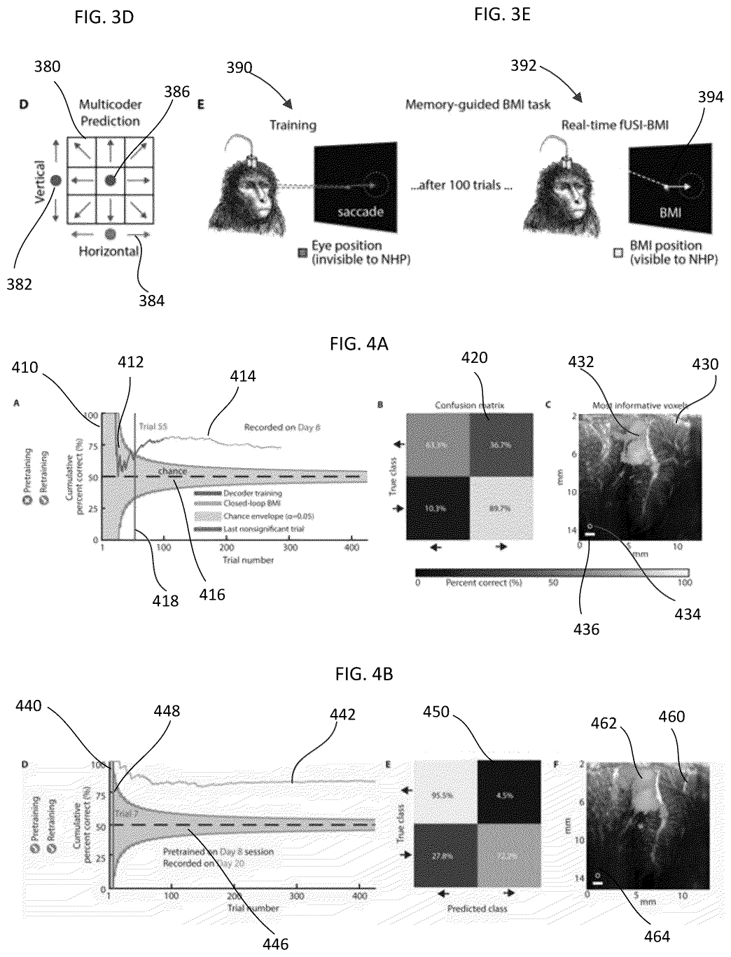

In the tests, coronal fUS imaging planes were used for the monkey P and the monkey L. A coronal slice from an MRI atlas showed the approximate field of view for the fUS imaging plane. 24×24 mm (inner dimension) chambers were placed surface normal to the skull of the test monkeys P and L above a craniotomy (black square). FIG. 3A shows a series of images that were acquired for the tests. A first image 310 shows a coronal field of view of the brain 222 superimposed on a coronal image from an MRI standardized monkey brain atlas. A box 312 shows the position of the transducing surface 232 of the transducer 216 for the tests. The other images in FIG. 3A are grouped for the NHP test subject P and the NHP test subject L. An image 314 shows a top down representation of the brain of the test subject monkey P. A line 316 represents the first plane that an ultrasound image was taken from the test subject monkey P. A functional ultrasound image 318 shows the first plane represented by the line 316 in the image 314.

An image 320 shows a top down representation of the brain of the test subject monkey L. A line 322 represents the second plane that an ultrasound image was taken from and a line 324 represents the third plane that an ultrasound image was taken from. An ultrasound image 326 was generated from the second plane in the two target task performed by the test subject monkey L. An ultrasound image 328 was generated from the third plane in the eight target task performed by the test subject monkey L.

The ultrasound transducer 216 was positioned to acquire a consistent coronal plane across different sessions as shown by the line 316 on the image 314 and the lines 322 and 324 for the other two planes in the images 326 and 328. The vascular maps in each of the images 318, 326, and 328 show the mean power Doppler image from a single imaging session. The three imaging planes shown in images 318, 326, and 328 were chosen for good decoding performance in a pilot offline dataset. Anatomical labels were inserted in the images 318, 326 and 328.

FIG. 3B shows the process for the first tests that involve the memory-guided saccade task for two eye movement directions. The non-human primate (NHP) test subject 220 had the transducer 216 attached as explained above. A trial started with the NHP test subject 220 fixating on a center fixation cue on the display 218 (330). A target cue 350 was flashed in a peripheral location (312). The position of the eye of the NHP test subject as determined by the eye motion sensor 236 in FIG. 2A is represented by a box 352. During a memory period (314), the NHP test subject 220 continued to fixate on the center cue 350 and planned an eye movement to the peripheral target location. Once the fixation cue was extinguished, the NHP test subject 220 performed a saccade to the remembered location (316) and maintained fixation on the peripheral location (318) before receiving a liquid reward (320). The fixation, memory, and hold periods were subject to ±500 ms of jitter (uniform distribution) to prevent the NHP test subject from anticipating task state changes. The peripheral cue was chosen from two or eight possible target locations depending on the specific experiment.

FIG. 3C is a diagram showing the feedback process and data collection in the two direction eye movement test. As shown in FIG. 3C, the transducer 216 collected ultrasound data from the brain 222 of the test subject. The raw data was sampled and beam formed to functional ultrasound images (360). Real-time 2 Hz functional images were streamed to the linear decoder 276 that controlled the behavioral task. The decoder used the last 3 fUS images 362 (1.5 seconds) of the memory period to make a prediction. A task control routine 364 determined whether the prediction was correct based on the action of the test subject. If the prediction was correct, the data from that prediction was added to a training set 370. The decoder 276 was retrained after every successful trial from the training set 370. The training set 370 consisted of images from trials from the current session 372 and/or images from a previous fUS-BMI session 374.

FIG. 3D shows a chart 380 representing a multi-coder algorithm for the eight movement direction task. For predicting eight movement directions, a vertical component 382 includes three positions (up, middle, or down) and a horizontal component 384 includes three positions (left, middle, or right). The vertical and horizontal components 382 and 384 were separately predicted and then combined to form an eight position fUS-BMI prediction 386.

FIG. 3E shows the test process for the memory-guided BMI task. The BMI task is the same as the memory-guided saccade task except that the movement period is controlled by the brain activity (via fUS-BMI) rather than eye movements. A series of eye-movement trials was conducted (390). After 100 successful eye-movement trials, the trained fUS-BMI controlled the movement prediction (closed-loop control) (392). During the closed-loop mode, the NHP test subject had to maintain fixation on the center fixation cue until reward delivery. If the BMI prediction was correct and the NHP test subject held fixation correctly, the NHP test subject received a large liquid reward (1 sec; ˜0.75 mL). If the BMI prediction was incorrect, but the NHP test subject held fixation correctly, the NHP received a smaller liquid reward (100 ms; ˜0.03 mL). In this example, the eye position of the test subject was not visible on the display, but the BMI-controlled cursor position 394 on the display 218 was visible to the test subject.

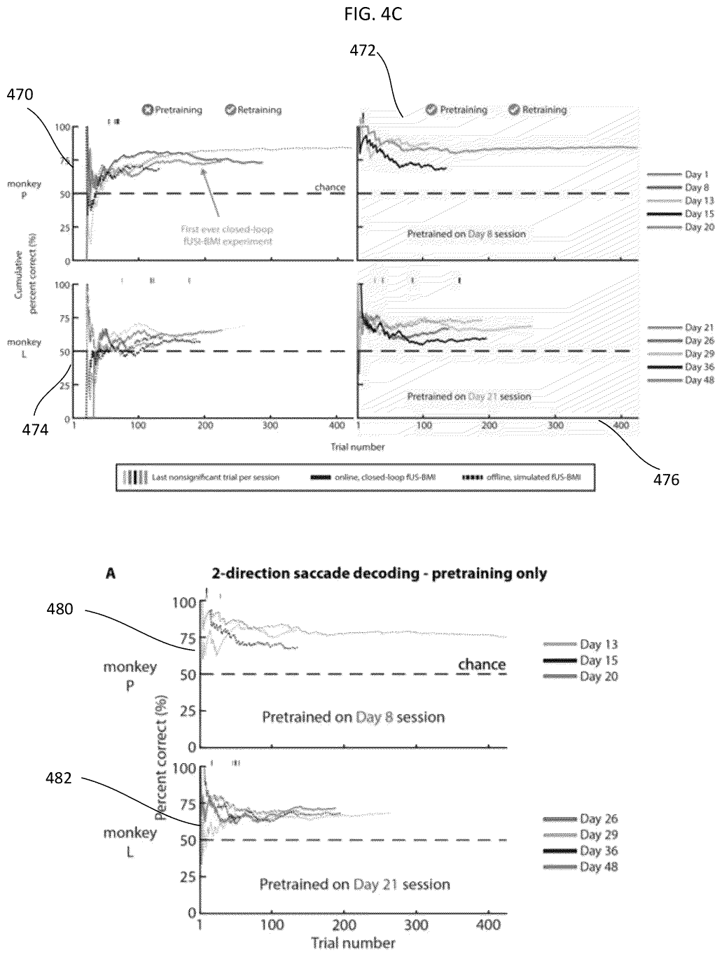

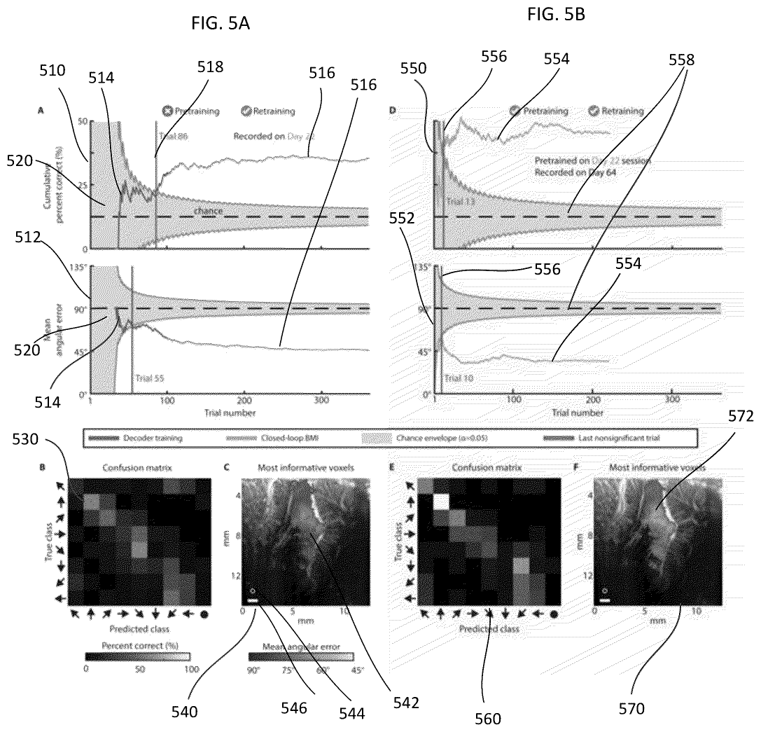

FIG. 4A shows results of the testing for decoding two saccade direction movement task for the test subject monkey P. A graph 410 shows the mean decoding accuracy as a function of the trial number. A trace 412 represents fUS-BMI training where the NHP test subject controlled the task using overt eye movements. The BMI performance shown in the plot 412 was generated post hoc with no impact on the real-time behavior. A trace 414 represents trials under fUS-BMI control where the monkey test subject maintained fixation on the center cue and the movement direction was controlled by the fUS-BMI. A shaded area 416 represents the chance envelope-95% binomial distribution. A line 418 represents the last nonsignificant trial.

FIG. 4A also shows a confusion matrix 420 of decoding accuracy across entire session represented as percentage (rows add to 100%). An ultrasound image 430 shows a shaded area 432 that shows the most informative voxels when performing the task. The ultrasound image 430 is a result of searchlight analysis represents the 10% voxels with the highest decoding accuracy in the shaded area 432 (threshold is q<=1.66e-6). A circle 434 represents the 200 μm searchlight radius used. A bar 436 is a 1 mm scale bar.

FIG. 4B shows results of the testing for decoding two saccade direction movement task for the test subject monkey P where the fUS-BMI decoder was pretrained using data collected from a previous session. In this example, the previous session was conducted on day 8, while the testing occurred in a session on day 20. A graph 440 shows the mean decoding accuracy as a function of the trial number. A trace 442 represents the BMI performance generated post hoc with no impact on the real-time behavior. The trace 442 represents trials under fUS-BMI control where the monkey maintained fixation on the center cue and the movement direction was controlled by the fUS-BMI. A shaded area 446 represents the chance envelope—95% binomial distribution. A line 448 represents the last nonsignificant trial, which is much more recent with the pretraining than that in the graph 410 in FIG. 4A.

FIG. 4B also shows a confusion matrix 450 of decoding accuracy across entire session represented as percentage (rows add to 100%). An ultrasound image 460 shows a shaded area 462 that shows the most informative voxels when performing the task. The ultrasound image 460 is a result of searchlight analysis represents the 10% voxels with the highest decoding accuracy in the shaded area 432 (corresponding to a threshold of q<=2.07e-13.). A circle 464 represents the 200 μm searchlight radius used.

FIG. 4C shows a series of graphs 470, 472, 474, and 476 that show performance across sessions for decoding two saccade directions for the two test subject monkeys. The graphs 470, 472, 474, and 476 shows mean decoder accuracy during each session for monkey P (graphs 470 and 472) and monkey L (graphs 474 and 476). In the graphs 470, 472, 474, and 476, the solid lines are real-time results while fine-dashed lines are simulated sessions from post hoc analysis of real-time fUS imaging data. Vertical marks above each plot represent the last nonsignificant trial for each session. The day number is relative to the first fUS-BMI experiment. The coarse-dashed horizontal black line in each of the graphs 470, 472, 474, and 476 represents chance performance.

The graph 470 represents the sessions conducted on days 1, 8, 13, 15, and 20 for the monkey P using the closed-loop BMI decoder. The graph 472 represents the results from days 13, 15, and 20 for the monkey P using pre-training data from the session on day 8. The graph 474 represents the results of the sessions conducted on days 21, 26, 29, 36, and 40 for the monkey L. The graph 476 represents the results of the sessions conducted on days 29, 36, and 40 based on pre-training data from the session on day 21.

After building a preliminary training set of 20 trials for the two direction task, the accuracy of the decoder 276 was tested on each new trial in a training mode not visible to the NHP test subject. This accuracy is plotted as the plot 412 in the graph 410 in FIG. 4A. After 100 trials, the BMI was switched from training to closed-loop decoding where the NHP test subject now controlled the task direction using his movement intention. The movement intention was determined via brain activity detected by the fUS-BMI decoder 276 in the last 3 fUS images of the memory period. The accuracy was shown by the plot 414 in the graph 410. At the conclusion of each trial, the NHP test subject received visual feedback of the fUS-BMI prediction. In the second closed-loop 2-direction session, the decoder reached significant accuracy (p<0.01; 1-sided binomial test) after 55 training trials and improved in accuracy until peaking at 82% accuracy at trial 114 as shown in the graph 410. The example decoder 276 predicted both directions well above chance level, but displayed better performance for rightward movements as shown in the confusion matrix 420. To understand which brain regions were most important for the decoder performance, the searchlight analysis with a 200 μm, i.e., 2 voxel, radius was performed to produce the image 430. The Dorsal LIP and Area 7a of the brain 222 contained the voxels most informative for decoding intended movement direction.

An ideal BMI needs very little training data and no retraining between sessions. Known electrode-based BMIs generally are quick to train on a given day but need to be retrained on new data for each new session due to their inability to record from the same neurons across multiple days. Due to a wide field of view, fUS neuroimaging can image from the same brain regions over time, and therefore is a desirable technique for stable decoding across many sessions. This was shown by retraining the fUS-BMI decoder 276 using previously recorded session data. The decoder was then tested in an online experiment as explained above. To perform this pretraining, the data from imaging plane of the previous session was aligned to the imaging plane of the current session as shown in FIG. 8. An image 810 from a day 1 session was added to an image 812 from a day 64 session. The images 810 and 812 were overlaid to produce a pre-registration alignment image 820. Rigid-body registration was applied to produce an image 822 of post-registration alignment.

Semi-automated intensity-based rigid-body registration was used to find the transform from the previous session to the new imaging plane. The registration error is shown in the overlay image 820 where a shaded area 830 represents the old session (Day 1) and a shaded area 832 represents the new session (Day 64). This 2D image transform was applied to each frame of the previous session, and the aligned data was saved. This semi-automated pre-registration process took less than 1 minute. To pretrain the model, the fUS-BMI computer 212 automatically loaded this aligned dataset and trained the initial decoder. The fUS-BMI reached significant performance substantially faster as shown by the graph 440 in FIG. 4B when pretraining was applied. The fUS-BMI achieved significant accuracy at trial 7, approximately 15 minutes faster than the example session without pretraining.

To quantify the benefits of pretraining upon fUS-BMI training time and performance, the fUS-BMI performance across all sessions was compared when (a) using only data from the current session versus (b) pretraining with data from a previous session. For all real-time sessions that used pretraining, a post hoc (offline) simulation of the fUS-BMI results without using pretraining was created. For each simulated session, the recorded data was passed through the same classification algorithm used for the real-time fUSI-BMI but did not use any data from a previous session.

A series of tests were performed using only data from the current session to assess the effectiveness of the pre-trained training set. In these tests, the mean decoding accuracy reached significance (p<0.01; 1-sided binomial test) at the end of each online, closed-loop recording session (2/2 sessions monkey P, 1/1 session monkey L) and most offline, simulated recording sessions (3/3 sessions monkey P, 3/4 sessions monkey L) as shown in the graphs in FIG. 4C. For monkey P, decoder accuracies reached 75.43±2.56% (mean±SEM) and took 40.20±2.76 trials to reach significance. For monkey L, decoder accuracies reached 62.30±2.32% and took 103.40±23.63 trials to reach significance.

A series of tests were performed for pretraining with data from a previous session. In these tests, the mean decoding accuracy reached significance at the end of each online, closed-loop recording session (3/3 sessions monkey P, 4/4 session monkey L) as shown in the graph 440 in FIG. 3B. Using previous data reduced the time to achieve significant performance (100% of sessions reached significance sooner, monkey P-36-43 trials faster; monkey L-15-118 trials faster). The performance at the end of the session was not statistically different from performance in the same sessions without pretraining (paired t-test, p<0.05). For monkey P, accuracies reached 80.21±5.61% and took 9.00±1 trials to reach significance. For monkey L, accuracies reached 66.78±2.79% and took 71.00±28.93 trials to reach significance. Assuming no missed trials, pre-training decoders shortened training by 10-45 minutes. The effects of not using any training data from the current session, i.e., using only the pretrained model was also simulated as shown in the graphs 480 and 482. The graph 480 shows the accuracy results of closed-loop, real-time decoding of movement directions using a pretrained model (based on day 8) only for the monkey P for sessions on days 13, 15, and 20. The graph 482 shows the accuracy results of closed-loop, real-time decoding of movement directions using a pretrained model (based on day 21) only for the monkey L for sessions on days 26, 28, 36, and 48. There was no statistical difference between the pretrained models with current session training data and without current session training data for accuracy or number of trials to significance for either NHP test subject for the performance of two direction saccade decoding using only the pretrained model.

These results show that two directions of movement intention may be decoded on line. The NHP test subjects could control the task using the example fUS-BMI. Pretraining using data from a previous session data greatly reduced, or even eliminated, the amount of new training data required in a new session.

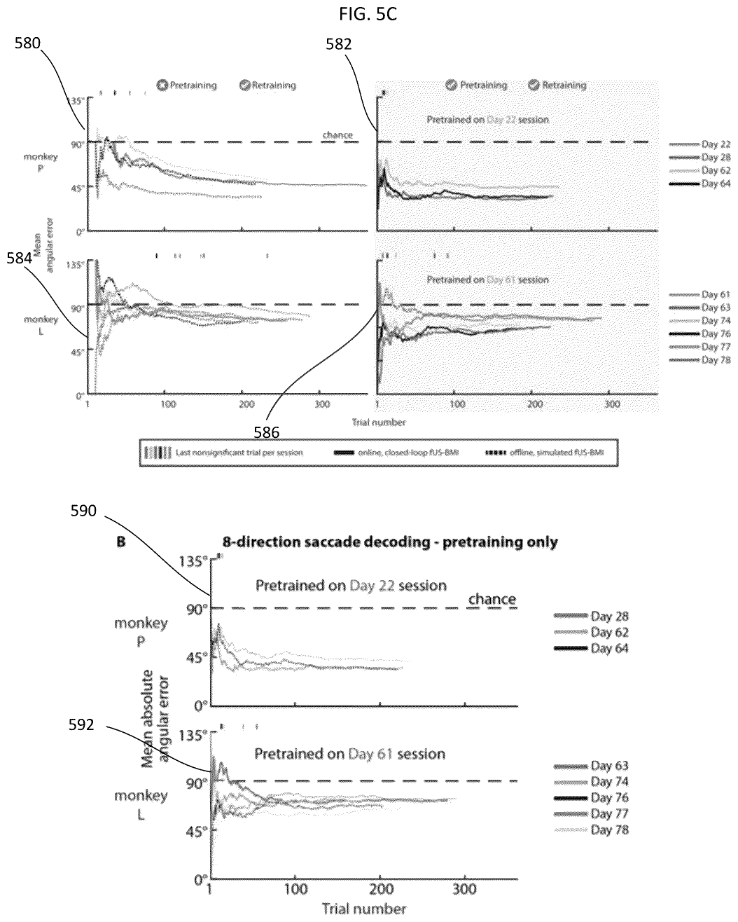

FIG. 5A shows results from example sessions testing the decoding of eight saccade directions. A graph 510 shows the mean decoding accuracy as a function of the trial number and a graph 512 shows the mean absolute angular error as a function of trial number. A plot 514 represents fUS-BMI training where the NHP test subject controlled the task using overt eye movements. The BMI performance shown here was generated post hoc tested on each new trial with no impact on the real-time behavior. A plot 516 represents trials under fUS-BMI control where the NHP test subject maintained fixation on the center cue and the movement task direction was controlled by the fUS-BMI. A line 518 represents the last non-significant trial. A shaded area 520 represents 90% binomial or permutation test distribution.

FIG. 5A also shows a confusion matrix 530 of final decoding accuracy across an entire session represented as percentage (rows add to 100%). The horizontal axis plots the predicted movement (predicted class) and the vertical axis the matching directional cue (true class). An image 540 is a searchlight analysis with an area 542 that represents the 10% of voxels with the lowest mean angular error (threshold is q≤2.98e-3). A circle 544 represents a 200 μm searchlight radius and a bar 546 is a 1 mm scale bar.

FIG. 5B shows the results from tests where the example fUS-BMI was pretrained on data from day 22 and updated after each successful trial. A graph 550 shows the mean decoding accuracy as a function of the trial number and a graph 552 shows the mean absolute angular error as a function of trial number. A plot 554 represents a fUS-BMI that was pretrained on data from a session conducted on day 22 where the NHP test subject maintained fixation on the center cue and the movement task direction was controlled by the fUS-BMI. A line 556 represents the last non-significant trial. A shaded area 558 represents 90% binomial or permutation test distribution.

FIG. 5B also shows a confusion matrix 560 of final decoding accuracy across an entire session represented as percentage (rows add to 100%). The horizontal axis plots the predicted movement (predicted class) and the vertical axis the matching directional cue (true class). An image 570 is a searchlight analysis with an area 572 that represents the 10% of voxels with the lowest mean angular error (threshold is q≤8e-5).

FIG. 5C is shows a series of graphs 580, 582, 584, and 586 that show performance across sessions for decoding eight saccade directions. The graphs 580, 582, 584, and 586 shows mean angular error during each session for monkey P (graphs 580 and 582) and monkey L (graphs 584 and 586). In the graphs 580, 582, 584, and 586, the solid lines are real-time results while the fine-dashed lines are simulated sessions from post hoc analysis of real-time fUS imaging data. Vertical marks above each plot represent the last nonsignificant trial for each session. The day number is relative to the first fUS-BMI experiment. The coarse-dashed horizontal black line in each of the graphs 580, 582, 584, and 586 represents chance performance.

The graph 580 represents the sessions conducted on days 22, 28, 62, and 64 for the monkey P using the closed-loop BMI decoder. The graph 582 represents the results from days 22, 28, 62, and 64 for the monkey P using pre-training from the session on day 22. The graph 584 represents the results of the sessions conducted on days 61, 63, 75, 76, 77, and 78 for the monkey L. The graph 586 represents the results of the sessions conducted on days 61, 63, 75, 76, 77, and 78 for the monkey L based on pre-training from the session on day 61.

The tests conducted for online decoding of eight eye movement directions demonstrate similar performances could be achieved, but online and closed-loop, for a fUS-BMI decoding eight movement directions in real time. A “multicoder” architecture was used where the vertical (up, middle, or down) and horizontal (left, middle, or right) components of intended movement were predicted separately and then combined those independent predictions to form a final prediction (e.g., up and to the right) as shown in the test set up in FIG. 3B. In the first 8-direction experiment, the decoder reached significant accuracy (p<0.01; 1-sided binomial test) after 86 training trials and improved until plateauing at 34-37% accuracy as shown in the graph 510 in FIG. 5A, compared to 12.5% chance level, with most errors indicating directions neighboring the cued direction as shown in the confusion matrix 530. To capture this extra information about proximity of each prediction to the true direction, the mean absolute angular error was examined. The fUS-BMI reached significance at 55 trials and steadily decreased the mean absolute angular error to 45° by the end of the session as shown in the graph 512. Compared to the most informative voxels for the 2-target eye decoder, a larger portion of the LIP, including the ventral LIP, contained the most informative voxels for decoding eight directions of movement as shown in the image 540.

Tests were conducted to determine whether pretraining would aid the 8-target decoding similar to pretraining the 2-target decoder. As before, pretraining reduced the number of trials required to reach significant decoding as shown in the graphs 550 and 552 in FIG. 5B. The fUS-BMI reached significant accuracy at trial 13, approximately 25 minutes earlier than using only data from the current session for training as shown in the graph 550. The mean decoder accuracy reached 45% correct with a final mean absolute angular error of 34°, which was better than the performance achieved in the example session without pretraining. The searchlight analysis indicated the same regions within the LIP provided the most informative voxels for decoding as shown in the image 570 for both the example sessions with and without pretraining. Notably, the fUS-BMI was pretrained on data from 42 days before the current session. This demonstrates that the fUS-BMI can remain stable over at least 42 days. This further demonstrates that the same imaging plane may be consistently located and that mesoscopic PPC populations consistently encode for the same directions on the time span of more than a month.

Tests were conducted using only data from the current session as shown in the graphs 510 and 512 in FIG. 5A. The mean decoder accuracy reached significance by the end of all real-time (2/2) and simulated sessions (8/8). The mean absolute angular error for monkey P reached 45.26±3.44° and the fUS-BMI took 30.75±12.11 trials to reach significance. The mean absolute angular error for monkey L reached 75.06±1.15° and the fUS-BMI took 132.33±20.33 trials to reach significance.

The results from pretraining with data from a previous session were shown in the graphs 582 and 586 in FIG. 5C. The mean decoder accuracy reached significance by the end of all real-time (6/6) and simulated sessions (2/2). The fUS-BMI reached significant decoding earlier for most sessions compared to simulated post hoc data; 5/5 faster monkey L; 2/3 faster monkey P (third session reached significance equally fast). For monkey P, the pretrained decoders reached significance 0-51 trials faster and for monkey L, the pretrained decoders reached significance 66-132 trials faster. For most sessions, this shortened training by up to 45 minutes. The performance at the end of each session was not statistically different from performance in the same session without pretraining (paired t-test, p<0.05). The mean absolute angular error for monkey P reached 37.82°±2.86° and the fUS-BMI took 10.67±1.76 trials to reach significance. The mean absolute angular error for monkey L reached 71.04°±2.29° and the fUS-BMI took 42.80±17.05 trials to reach significance.

The effects of not using any training data from the current session, i.e., using only the pretrained model was also simulated as shown in the graphs 590 and 592 in FIG. 5C. The graph 590 shows the accuracy results of closed-loop, real-time decoding of movement directions using a pretrained model (based on day 22) only for the monkey P for sessions on days 28, 62, and 64. The graph 592 shows the accuracy results of closed-loop, real-time decoding of movement directions using a pretrained model (based on day 61) only for the monkey L for sessions on days 63, 74, 76, 77, and 78. There was no statistical difference between the pretrained models with and without current session training data for accuracy, mean absolute angular error, or number of trials to significance for either NHP test subject performing the 8-direction saccade task.

These results show that eight directions of movement intention may be decoded in real-time at well above chance level. This demonstrates that the example online, closed-loop fUS-BMI system is sensitive enough to detect more than differences between contra- and ipsilateral movements. The directional encoding within PPC mesoscopic populations is stable across more than a month, allowing the reduction or even elimination of the need for new training data.

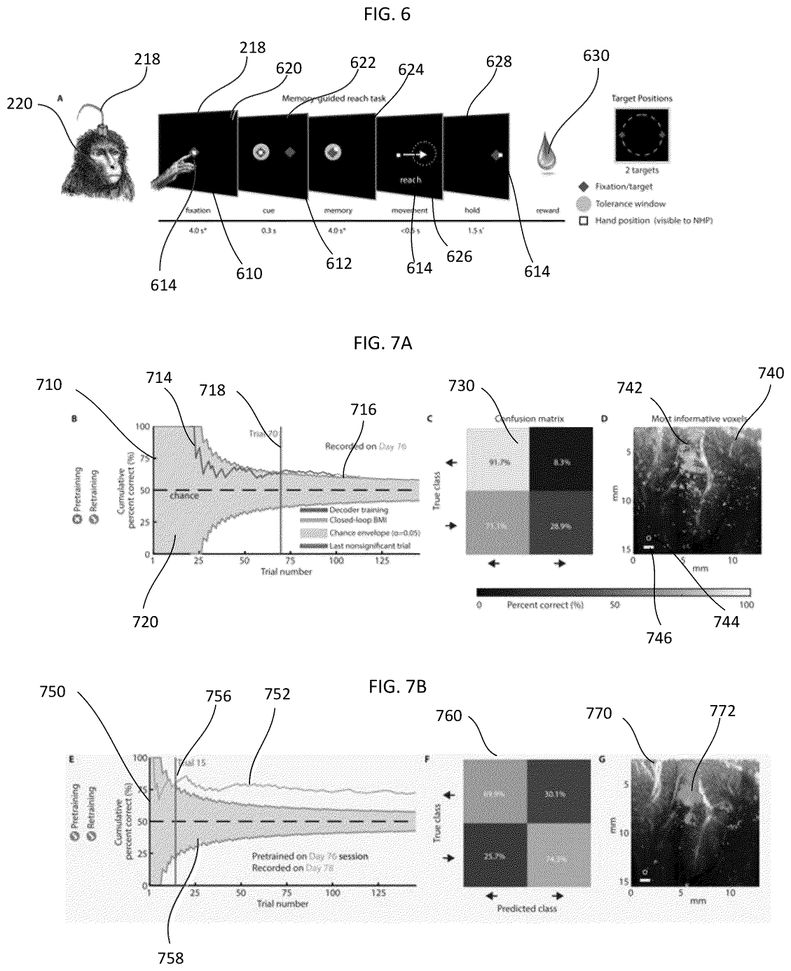

Another strength of fUS neuroimaging is its wide field of view capable of sensing activity from multiple functionally diverse brain regions, including those that encode different movement effectors, e.g., hand and eye. To test this, intended hand movements to two target directions (reaches to the left or right for monkey P) were decoded in addition to the previous results decoding eye movements as explained in relation to the testing process in FIG. 6. FIG. 6 shows a memory-guided two direction reach task for the test subject 220 with the implanted transducer 216. The NHP test subject 220 performed a similar task where the test subject had to maintain touch on a center dot and touch the peripheral targets during the training. In this scenario, hand movements were recorded to train the fUS-BMI system. After the training period, the test subject monkey controlled the task using the fUS-BMI system while keeping his hand on the center fixation cue.

In this test, the monkey P served as the test subject 220. The set up for the memory-guided reach task is identical to the memory-guided saccade task shown in FIG. 3B with all fixation or eye movements being replaced by maintaining touch on the screen 218 and reach movements respectively. After the training period, the monkey test subject 220 controlled the task using the fUS-BMI while keeping his hand on a center fixation cue 610 (620). The same imaging plane was used for eye movement decoding, which contained both LIP (important for eye movements) and MIP (important for reach movements). A target cue 612 was flashed in a peripheral location (622). During a memory period (624), the test subject 220 continued to fixate on the center cue 610 and planned hand movement to the peripheral target location. Once the fixation cue was extinguished, the NHP test subject 220 moved the hand to the remembered location (626) and held the hand on the peripheral location (628) before receiving a liquid reward (630). A square 614 represents the hand position and is visible to the monkey.

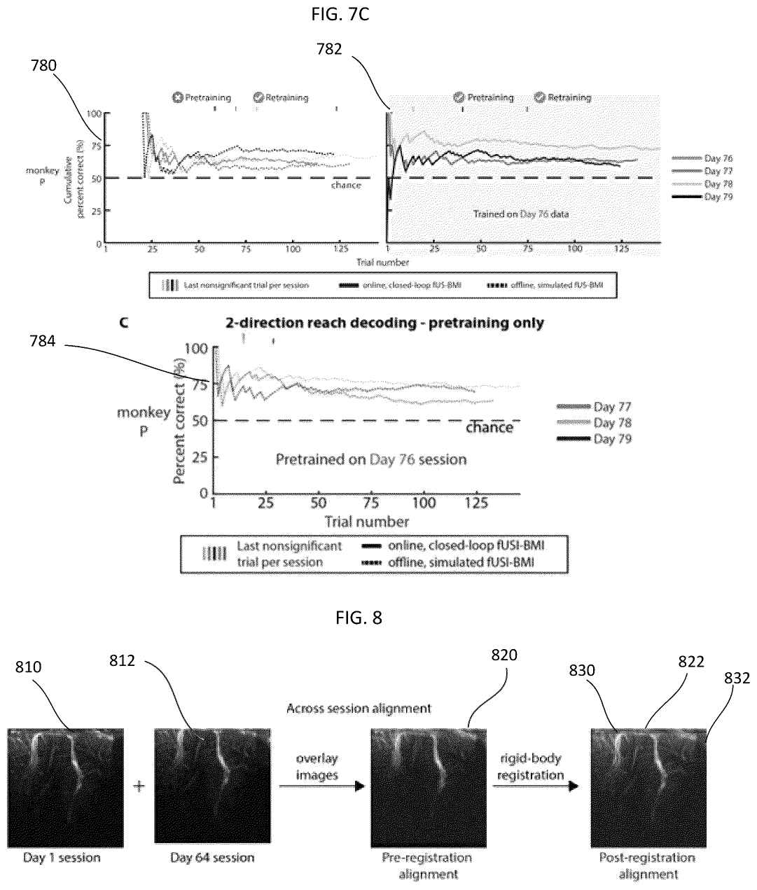

FIG. 7A shows results from example sessions decoding the two direction reach task test shown in FIG. 6. A graph 710 shows the decoding accuracy as a function of the trial number. A plot 714 represents fUS-BMI training where the NHP test subject controlled the task using overt hand movements. The BMI performance shown here was generated post hoc tested on each new trial with no impact on the real-time behavior. A plot 716 represents trials under fUS-BMI control where the NHP test subject maintained fixation on the center cue and the movement task direction was controlled by the fUS-BMI. A line 718 represents the last non-significant trial. A shaded area 720 represents 90% binomial or permutation test distribution.

FIG. 7A also shows a confusion matrix 730 of final decoding accuracy across an entire session represented as percentage (rows add to 100%). The horizontal axis plots the predicted movement (predicted class) and the vertical axis the matching directional cue (true class). An image 740 is a searchlight analysis with areas 742 that represents the 10% of voxels with the lowest mean angular error (threshold is q≤3.05e-3). A circle 744 represents a 200 μm searchlight radius and a bar 746 is a 1 mm scale bar.

In the example session using only data from the current session shown in FIG. 7A, it took 70 trials to reach significance and achieved a mean decoder accuracy of 61.3%. The decoder predominately guessed left as shown in the confusion matrix 730. Two foci within the dorsal LIP and scattered voxels throughout Area 7a and the temporo-parietal junction (Area Tpt) contained the most informative voxels for decoding the two movement directions as shown in the image 740.

FIG. 7B shows the results from tests where the example fUS-BMI was pretrained on data from day 76 and updated after each successful trial. A graph 750 shows the decoding accuracy as a function of the trial number. A plot 752 represents fUS-BMI that was pretrained on data from a session conducted on day 76 where the NHP test subject maintained fixation on the center cue and the movement task direction was controlled by the fUS-BMI. A line 756 represents the last non-significant trial. A shaded area 758 represents 90% binomial or permutation test distribution.

FIG. 7B also shows a confusion matrix 760 of final decoding accuracy across an entire session represented as percentage (rows add to 100%). The horizontal axis plots the predicted movement (predicted class) and the vertical axis the matching directional cue (true class). An image 770 is a searchlight analysis with an area 772 that represents the 10% of voxels with the lowest mean angular error (threshold is q≤ 6.47c-5).

As with the saccade decoders, pretraining of the movement decoder significantly shortened training time. In some cases, pretraining rescued a “bad” model. For example, the example session using only current data as shown in the confusion matrix 730 in FIG. 7A displayed a heavy bias towards the left. When this example session was used to pretrain the fUS-BMI a few days later, the new model made balanced predictions as shown in the confusion matrix 760. The searchlight analysis for this example session revealed that the same dorsal LIP region from the example session without pretraining contained the majority of the most informative voxels as shown in the image 770. MIP and Area 5 also contained a few patches of highly informative voxels.

FIG. 7C shows a graph 780 of the cumulative percentage correct for the monkey P for sessions conducted on days 76, 77, 78 and 79. A graph 782 shows the cumulative percentage correct for monkey P for sessions based on pretraining from day 76 day. In the graphs 780 and 782, the solid lines are real-time results while fine-dashed lines are simulated sessions from post hoc analysis of real-time fUS imaging data. Vertical marks above each plot represent the last nonsignificant trial for each session. The day number is relative to the first fUS-BMI experiment. The coarse-dashed horizontal line in the graphs 780 and 782 represents chance performance.

Using only data from the current session as shown in the graph 780, the mean decoder accuracy reached significance by the end of each session (1 real-time and 3 simulated) as shown in the plots in the graph 780. The performance reached 65±2% and took 67.67±18.77 trials to reach significance.

When testing pretraining with data from a previous session as shown in the graph 782, the mean decoder accuracy reached significance by the end of each session (3 real-time). The performance of the monkey P reached 65±4% and took 43.67±17.37 trials to reach significance. For two of the three real-time sessions, the number of trials needed to reach significance decreased with pretraining (−2-46 trials faster; 0-16 minutes faster). There was no statistical difference in performance between the sessions with and without pretraining (paired t-test, p<0.05). The effects of not using any training data from the current session, i.e., using only the pretrained model were also studied. A graph 784 shows the percentage correct for monkey P for sessions conducted on days 77, 78, and 79 using only the pre-trained model. There was no statistical difference between the pretrained models with and without current session training data for accuracy or number of trials to significance.

These results agree with the previous results that not only eye movements may be decoded, but also reach movements may be decoded. As with the eye movement decoders, the fUS-BMI may be retrained using data from a previous session reduce, or even eliminate, the need for new training data.

Additional tests were performed to determine whether mesoscopic regions of PPC subpopulations that were robustly tuned to individual movement directions were stable across time. Data was collected from the same coronal plane from two NHP test subjects across many months performing the eight direction memory guided saccade task described above. The example BMI decoder was trained on data from a previous session. The BMI decoder was tested on the ability to predict movement direction on other sessions from that same plane without any retraining of the decoder. The analysis was repeated using each session as the training set and testing all the other sessions.

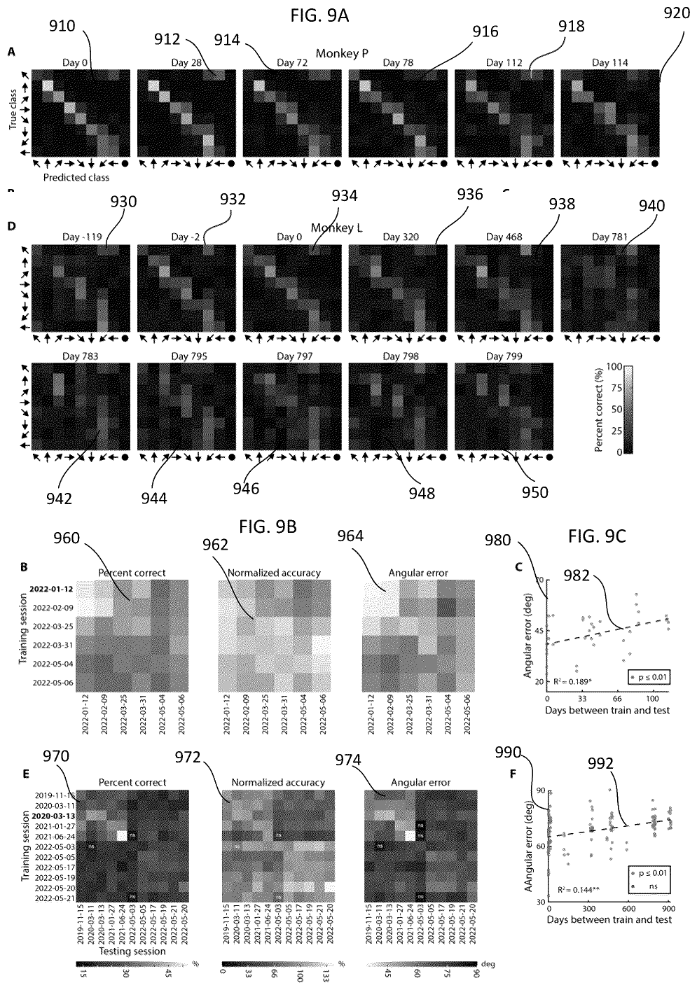

FIG. 9A shows a set of confusion matrices 910, 912, 914, 916, 918, and 920 for test sessions from respective day 0, day 28, day 72, day 78, day 112, and day 114 for the test subject, Monkey P. FIG. 9A also shows a set of confusion matrices 930, 932, 934, 936, 938, 940, 942, 944, 946, 948, and 950 for test sessions from respective day-119, day-2, day 0, day 328, day 468, day 781, day 783, day 795, day 797, day 798, and day 799 for the test subject, Monkey L.

FIG. 9B shows a graph 960 that shows the percentage correct based on the training session data from the session conducted on Jan. 12, 2022 in comparison with subsequent sessions for the test subject, Monkey P. A graph 962 shows the normalized accuracy based on the training session data from the session conducted on Jan. 12, 2022 in comparison with subsequent sessions for the test subject, Monkey P. A graph 964 shows the angular error based on the training session data from the session conducted on Jan. 12, 2022 in comparison with subsequent sessions for the test subject, Monkey P. A graph 970 shows the percentage correct based on the training session data from the session conducted on Mar. 13, 2020 in comparison with other sessions for the test subject, Monkey L. A graph 972 shows the normalized accuracy based on the training session data from the session conducted on Mar. 13, 2020 in comparison with other sessions for the test subject, Monkey L. A graph 974 shows the angular error based on the training session data from the session conducted on Mar. 13, 2020 in comparison with other sessions for the test subject, Monkey L. The graphs 960, 962, 964, 970, 972, and 974 show decoder stability for training and testing on each session. The nonsignificant decoding performance, a was 0.01.

FIG. 9C shows a graph 980 plotting mean angular error as a function of days between the training and testing session for the test subject, Monkey P. A dashed line 982 represents a linear fit to data where *=p<10⁻², and **=p<10⁻⁴ with a R² value of 0.189. A graph 990 plots mean angular error as a function of days between the training and testing session for the test subject, Monkey L. A dashed line 992 represents a linear fit to data where *=p<10⁻², and **=p<10⁻⁴ with a R² value of 0.144. The R² value, or the coefficient of determination, is a measure between 0 and 1 of how well the linear fit models the data. The positive value of R² suggests there is a weak relationship between decoder performance and amount of time since the training session.

For Monkey P, the decoder performance remained significant even after more than 100 days between the training and testing sessions. All pairs of training and testing session for Monkey P showed significant decoding performance (p<10-5; 36/36 pairs) as shown by the graphs 960, 962, and 964. For Monkey L, the decoder performance remained significant across more than 900 days. Almost all pairs of training and testing session for Monkey L showed significant decoding performance (p<0.01; 117/121 pairs) as shown by the graphs 970, 972, and 974. Different training sessions had different decoding performance when tested on itself using cross-validation (diagonal of performance matrices), so w, the accuracy normalized to the training session's cross-validated accuracy, was examined. No clear differences between the absolute and normalized accuracy measures were observed. For Monkey L, the decoder trained on the Mar. 13, 2021 session performed the best for three directions (contralateral up, contralateral down, and ipsilateral down) in the training set and continued to decode these same three directions the best consistently throughout the test sessions as shown in the confusion matrices 930, 932, 934, 936, 938, 940, 942, 944, 946, 948, and 950. This pattern was observed consistently where the decoder could best predict certain directions, even when the training session had poor cross-validated performance by itself.

In both monkeys, temporally adjacent sessions had better performance as shown in the graphs 980 and 990. For Monkey L, the performance was clumped into two temporal groups (before and after May 3, 2023) where training on a session within its same temporal group provided the best performance. Physical changes in the imaging plane across time may explain the decrease in performance. There were out-of-plane alignment issues where the major blood vessels were very similar but mesovasculature would change. This out-of-plane difference was <400 μm across all recording sessions based upon comparing the fUSI imaging planes with anatomical MRIs of the brains of the test subjects. The results demonstrate that a decoder trained from data from a previous session could be used to successfully perform the task in a session occurring at least 900 days from the previous session.

To determine whether differences in vascular anatomy led to the decrease in decoder performance, the similarity of the vascular anatomy across time was examined using an image similarity metric, the complex-wavelet structural similarity index measure (CW-SSIM).

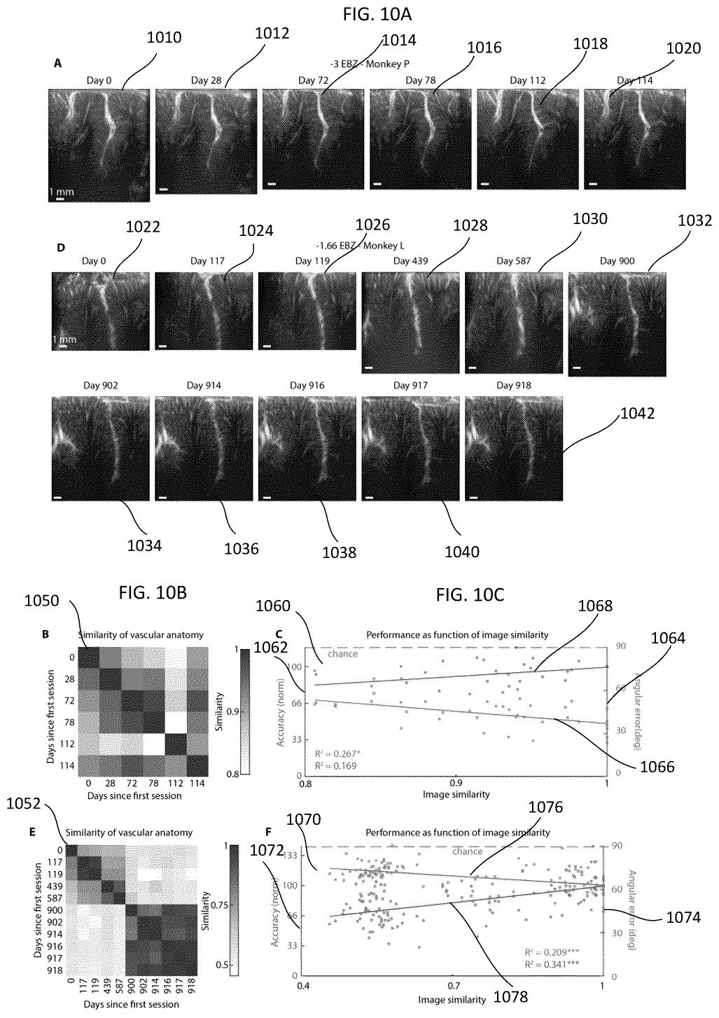

FIG. 10A shows images 1010, 1012, 1014, 1016, 1018, and 1020 of vascular anatomy for each recording session from the same recording slot in Monkey P. The images 1010, 1012, 1014, 1016, 1018, and 1020 are from respective day 0, day 28, day 72, day 78, day 112, and day 114 for Monkey P. Similarly, images 1022, 1024, 1026, 1028, 1030, 1032, 1034, 1036, 1038, 1040, and 1042 of vascular anatomy are from respective day 0, day 117, day 119, day 439, day 587, day 900, day 902, day 914, day 916, day 917, and day 918 for Monkey L. The images in FIG. 10A show that there are differences in vascular anatomy over different sessions.

FIG. 10B shows a chart 1050 of pair-wise similarity between different vascular images for Monkey P for days since the first session. FIG. 10B also shows a chart 1052 of pair-wise similarity between different vascular images for Monkey P for days since the first session.

FIG. 10C shows a graph 1060 showing performance as a function of image similarity for Monkey P. A left axis 1062 shows normalized accuracy. A right axis 1064 shows mean angular error. Each session is represented by a pair of blue and orange dots. A plot 1066 represents the linear fitted model between image similarity (measured by CW-SSIM) and angular error (axis 1062) with an R² of 0.169. A plot 1068 represents the linear fitted model between image similarity (measured by CW-SSIM) and normalized accuracy (axis 1062) with an R² of 0.267. Similarly, a graph 1070 shows performance as a function of image similarity for Monkey L. A left axis 1072 shows normalized accuracy. A right axis 1074 shows mean angular error. Each session is represented by a pair of blue and orange dots. The blue dots represent normalized accuracy (axis 1072). The orange dots represent the angular error (axis 1074). A plot 1076 represents the linear fitted model between image similarity (measured by CW-SSIM) and angular error (axis 1074) with an R² of 0.341. A plot 1078 represents the linear fitted model between image similarity (measured by CW-SSIM) and normalized accuracy (axis 1072) with an R² of 0.209. *=p<10-2, **=p<10-4, ***=p<10-6, where this represents the statistical significance of the linear fit. Together, plots 1068 and 1078 show that training and testing sessions that have higher image similarity have improved normalized accuracy. Similarly, plots 1066 and 1076 show that training and testing sessions have higher image similarity have reduced angular error.

The CW-SSIM clumps the vascular images into discrete groups matching the qualitative assessment of image similarity as shown in FIG. 10B that matched qualitative assessment of image similarity. The similarity grouping also matched the pairwise decoding performance grouping in Monkey L as shown in the graphs 970, 972, and 974. The decoder performance and image similarity were correlated as shown in FIG. 10C. As image similarity decreased between the training session and each test session, the decoder performance also decreased. This shows that the decrease in decoder performance resulted from changes in the imaging plane rather than drift in the tuning of each subpopulation.

The example closed-loop, online, ultrasonic BMI system and method may be applied to a next generation of minimally-invasive ultrasonic BMIs via the ability to decode more movement directions and stabilize decoders across more than a month.

The decoding of more movement directions was shown in the successful decoding of eight movement directions in real-time. Specifically, the two direction results were replicated using real-time online data and extended to the decoder to work for eight movement directions.

The stabilizing of the decoder across time was shown by electrode-based BMIs that are particularly adept at sensing fast changing (˜10 s of ms) neural activity from spatially localized regions (<1 cm) during behavior or stimulation that is correlated to activity in such spatially specific regions, e.g., M1 for motor and VI for vision. Electrodes, however, suffer from an inability to track individual neurons across recording sessions. Consequently, decoders based on data from electrodes are typically retrained each day. In the example system image-based BMIs were stabilized across more than a month and decode from the same neural populations with minimal, if any, retraining. This is a critical development that enables easy alignment of models from previous days to data from a new day. This allows decoding to begin while acquiring minimal to no new training data. Much effort has focused on reducing or eliminating re-training in electrode-based BMIs. However, these methods require identification of manifolds and/or latent dynamical parameters and collecting new data to align to these manifolds/parameters. Furthermore, some of the algorithms are not yet optimized for online use and/or are computationally expensive and difficult to implement. The example pre-trained decoder alignment algorithm leverages the intrinsic spatial resolution and field of view provided by fUS neuroimaging to simplify this process in a way that is intuitive, repeatable, and performant.

The present system may be improved in several ways. First, realigning the recording chamber and ultrasound transducer along the intraparietal sulcus axis would allow sampling from a larger portion of LIP and MIP. In the tests described herein, the chamber and probe were placed in a coronal orientation to aid anatomical interpretability. However, most of the imaging plane is not contributing to the decoder performance in these tests. Receptive fields are anatomically organized along anterior-posterior and dorsal-ventral gradients within LIP25. By realigning the recording chamber orthogonal to the intraparietal sulcus, sampling may be performed from a larger anterior-posterior portion of LIP with diverse range of directional tunings.

Second, the advent of 3D ultrafast volumetric imaging based on matrix or row-column array technologies will be capable of sensing changes in CBV from blood vessels that are currently orthogonal to the imaging plane. Additionally, 3D volumetric imaging can fully capture entire functional regions and sense multiple functional regions simultaneously. There are many regions which could fit inside a field of view of a 3D probe and contribute to a motor BMI, for example: PPC, primary motor cortex (M1), dorsal premotor cortex (PMd), and supplementary motor area (SMA). These areas encode different aspects of movements including goals, sequences, and expected value of actions. This is just one example of myriad advanced BMI decoding strategies that will be made possible by synchronous data across brain regions.

Third, another route for improved performance is using more advanced decoder models. In the example herein, linear discriminant analysis was used to classify the intended target of the NHP test subjects. Artificial neural networks (ANNs) are an option. For example, convolutional neural networks are tailor-made for identifying image characteristics. Recurrent neural networks and transformers use “memory” processes and may be particularly adept at characterizing the temporal structure of fUS time series data. A potential downside of ANNs is that they require significantly more data than the linear method presented here. However, the example methods for across session image alignment will allow for already-collected data to be aggregated and organized into a large data corpus. Such a data corpus should be sufficient for small to moderately sized ANNs. The amount of training data required may be further reduced by decreasing the feature counts of the ANNs. For example, the input layer dimensions may be reduced by restricting the image to features collected only from the task-relevant areas, such as LIP and MIP, instead of the entire image. ANNs additionally take longer to train (˜minutes instead of seconds) and require different strategies for online retraining. Due to ANNs taking substantially longer to train, a different strategy of training during the intertrial interval following any addition to the training set is necessary. For example, a parallel computing thread that retrained the ANN every 10-20 trials may be implemented and then the fUS-BMI could retrieve the latest trained model on each trial.

fUS neuroimaging has several advantages compared to existing BMI technologies. The large and deep field of view of fUS neuroimaging allows reliable recording from multiple cortical and subcortical regions simultaneously—and to record from the same populations in a stable manner over long periods of time. fUS neuroimaging is epidural, i.e., does not require penetration of the dura mater, substantially decreasing surgical risk, infection risk, and tissue reactions while enabling chronic imaging over long periods of time (potentially many years) with minimal, if any degradation, in signal quality. In the NHP studies, fUS neuroimaging has been able to image through the dura, including the granulation tissue that forms above the dura (several ˜mm) with minimal loss in sensitivity.

The example fUSI system with a pre-trained decoder is stable across multiple days, months or even years. Using conventional image registration methods, the decoder may be aligned across different recording sessions and achieve excellent performance without collecting additional training data. A weakness of this current fUS-BMI compared to current electrophysiology BMIs is poorer temporal resolution. Electrophysiological BMIs have temporal resolutions in the 10 s of milliseconds (e.g., binned spike counts). fUS can reach a similar temporal resolution (up to 500 Hz in this work) but is limited by the time constant of mesoscopic neurovascular coupling (˜seconds). Despite this neurovascular response acting as a low pass filter on each voxel's signal, faster fUS acquisition rates can measure temporal variation across voxels down to <100 ms resolution.

As the temporal resolution and latency of real-time fUS imaging improves with enhanced hardware and software, tracking the propagation of these rapid hemodynamic signals will enable improved BMI performance and response time. Beyond movement, many other signals in the brain may be better suited to the spatial and temporal strengths of fUS, for example, monitoring biomarkers of neuropsychiatric disorders.

As explained above, dorsal and ventral LIP contained the most informative voxels when decoding eye movements. This is consistent with previous findings that the LIP is important for spatially specific oculomotor intention and attention. Dorsal LIP, MIP, Area 5, and Area 7 contained the most informative voxels during reach movements. The voxels within the LIP closely match with the most informative voxels from the 2-direction saccade decoding, suggesting that the example fUS-BMI is using eye movement plans to build its model of movement direction. The patches of highly informative voxels within MIP and Area 5 indicate the example fUS-BMI are using reach-specific information.

The vast majority of BMIs have focused on motor applications, e.g., restoring lost motor function in people with paralysis. Closed-loop BMIs may restore function to other demographics, such as patients disabled from neuropsychiatric disorders. Depression, the most common neuropsychiatric disorder, affects an estimated 3.8% of people (280 million) worldwide. The utility of the fUS-BMI for motor applications allows easier comparison with existing BMI technologies. fUS-BMIs are an ideal platform for applications that require monitoring neural activity over large regions of the brain and long time scales, from hours to months. As demonstrated by the tests, fUS neuroimaging captures neurovascular changes on the order of a second and is also stable over more than 1 month. Combined with neuromodulation techniques such as focused ultrasound, not only these distributed corticolimbic populations may be recorded but also specific mesoscopic populations may be precisely modulated. Thus, the fUS-BMI using the techniques herein may be applied to a broader range of applications, including restoring function to patients suffering from debilitating neuropsychiatric disorders.

Visualization of human brain activity is crucial for understanding normal and aberrant brain function. Currently available neural activity recording methods are highly invasive, have low sensitivity, and cannot be conducted outside of an operating room. In order to facilitate functional ultrasound imaging (fUSI) for sensitive, large-scale, high-resolution neural imaging in an adult human skull, an example cranial implant with a polymeric skull replacement material with a sonolucent acoustic window compatible with a fUSI system to monitor adult human brain activity in a single individual is disclosed. Using an in vitro cerebrovascular phantom to mimic brain vasculature and an in vivo rodent cranial defect model, the fUSI signal intensity and signal-to-noise ratio through the example polymethyl methacrylate (PMMA) cranial implant of different thicknesses or a titanium mesh implant was evaluated. The testing showed that rat brain neural activity could be recorded with high sensitivity through a PMMA implant using a dedicated fUSI pulse sequence. An example custom ultrasound transparent cranial window implant was designed for an adult patient undergoing reconstructive skull surgery after traumatic brain injury. Testing showed that a fUSI system with the example custom implant could record brain activity in an awake human outside of the operating room. In a video game “connect the dots” task, mapping and decoding of task-modulated cortical activity in the test participant was demonstrated. In a guitar strumming task, additional task-specific cortical responses were mapped using the example custom interface. The tests showed that fUSI can be used as a high-resolution (200 μm) functional imaging modality for measuring adult human brain activity through an implant having an acoustically transparent cranial window.

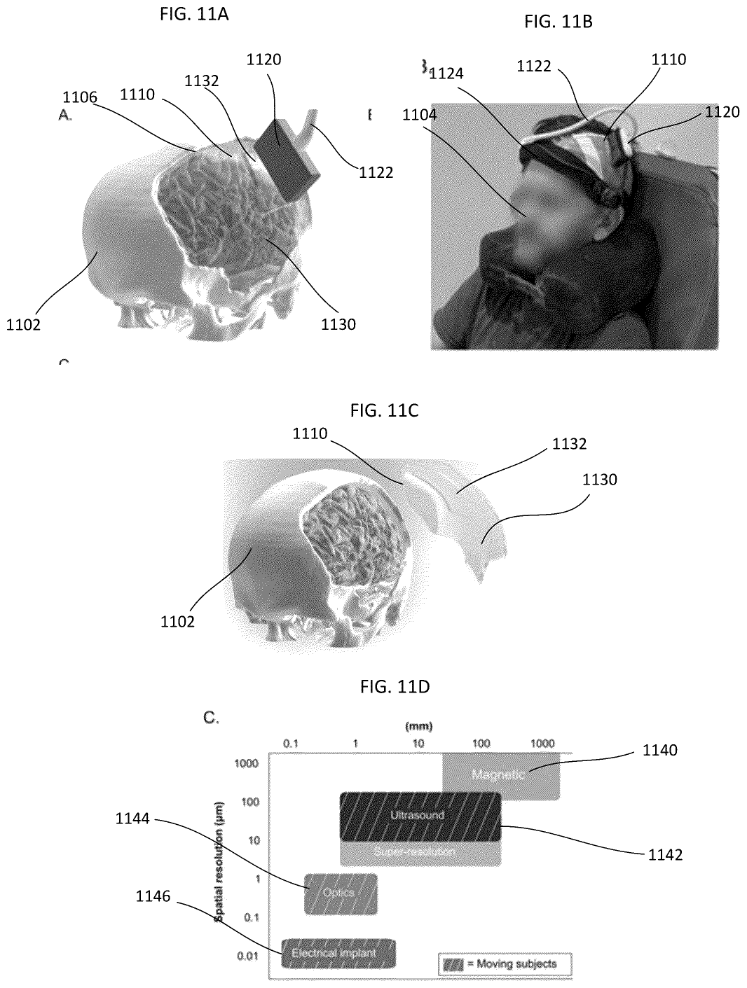

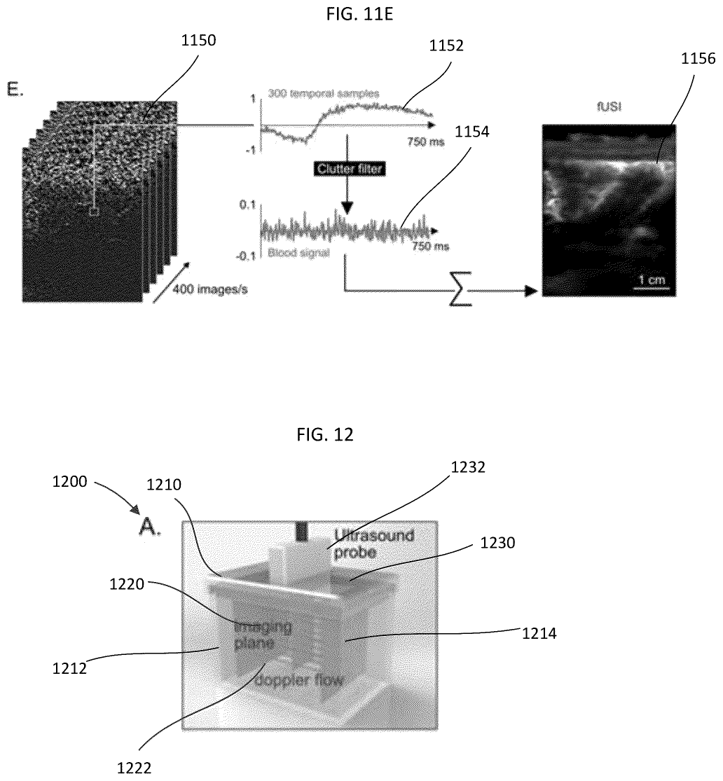

FIG. 11A is a close up cutaway perspective view of a skull 1102 with a customized cranial implant (CCI) 1110 that may be substituted for the chamber 104 in FIG. 1 for facilitating use of a fUSI transducer probe. In this example, the cranial implant 1110 may be customized for a particular patient. Customizing the cranial implant 1110 for a particular patient in this example includes designing the shape of the implant to fit the skull defect. The design is based upon previous anatomical imaging of the skull through imaging such as MRI. The design places a thinned portion constituting the sonolucent window over brain regions of interest. The thicknesses of the different parts of the implant itself are subject to safety considerations that limit minimum thickness/size. FIG. 11B is a perspective view of the customized cranial implant 1110 on a test subject 1104. As will be explained, the cranial implant 1100 is fabricated from sonolucent material and thus allows imaging from signals from a transducer probe 1120 similar to the probe 102 in FIG. 1. The cranial implant 1110 is shown on the skull 1102 with a craniotomy that removed a skull section leaving an opening 1106. In this example, the ultrasound probe 1120 is attached to the implant 1110 via custom-designed headband 1124 that holds the ultrasound probe 1120 in place against the scalp. The ultrasound probe 1120 includes transducer elements for an fUSI system such as that in FIG. 1. The transducer elements may comprise piezoelectric crystals (or semiconductor based transducer elements) to emit pulsed ultrasonic signals. The transducer elements may be organized as a single transducer or in an array of transducer elements. The ultra-sound probe 1120 is coupled to a cable 1122 that allows power and data signals to be communicated with an external fUSI system.

FIG. 11C is a perspective isolated view of the example cranial implant 1100. As shown in FIG. 11C, the implant 1110 includes a support section 1130 that is shaped to be inserted in the removed section 1106 of the skull 1102. The support section 1130 thus replaces the section 1106 removed from the skull 1102 via surgery. A window section 1132 is created that is generally thinner than the surrounding material of the support section 1130 to allow fUSI signals access to the brain of the patient 1104. In this example, the window section 1132 is a parallelogram shape that is positioned so relevant parts of the brain may be imaged. For example, the window section 1132 may be positioned relative to the support section 1130 to allow fUSI recording of the primary motor cortex, primary somatosensory cortex, and posterior parietal cortex of the brain. Alternatively, the window shape, location, and size may be designed for allowing fUSI recording over one or more of these areas or other areas of interest of the brain. fUSI recording may thus be accurately obtained through a cranial window defined by the window section 1132. As will be explained the location and size of the window section is determined through analysis of standard fMRI through the removed section 1106. Alternatively, the location and size of the window section may be determined through analysis of other imaging methods such as fUSI through the removed section 1106. Alternatively, the sonolucent window may include the entire support section of the cranial implant. In this instance, the thickness of the entire cranial implant is thin enough to allow fUSI recording over any part of the implant.

In this example, the example support section 1130 and window section 1132 of the cranial implant 1110 is fabricated from a polymeric skull replacement material such as polymethyl methacrylate (PMMA). Other polymeric skull replacement materials such as polyether ether ketone (PEEK) may be used for the cranial implant. Alternatively, other non-polymeric skull replacement materials may be used for the support section with a metallic (acoustically transparent) mesh, such as a titanium mesh, for the sonolucent window. The window section 1132 in this example is a 2 mm-thick 34×50 mm parallelogram-shaped sonolucent “window to the brain.” In this example, the implant window section 1132 sits above the primary motor cortex, primary somatosensory cortex, and posterior parietal cortex of the brain of the patient 1104 and allows fUSI recording of these areas. The remaining PMMA of the support section 1130 of the implant 210 is 4 mm thick. The implant 1110 provides sufficient mechanical performance to serve as a permanent skull replacement on the section 1106 removed from the skull 1102. The window section 1132 may be other thicknesses such as the thickness of a human skull e.g., approximately 1-10 mm in thickness.

The advantages of fUSI in comparison with other image techniques are shown in FIG. 11D, which is a graph of spatial resolution versus coverage for magnetic imaging, ultrasound imaging, optics imaging and electrical implants as represented respectively by bars 1140, 1142, 1144, and 1146. The graph in FIG. 11D also shows the ability to record moving subjects for each of the imaging techniques. As shown in FIG. 11D, electrical implants, optics and ultrasound imaging allow tracking moving objects with ultrasound imaging providing both spatial resolution and coverage.

FIG. 11E shows the process of ultrafast acquisition of ultrasound images 1150 to allow a fast temporal sampling of the brain signal as shown in a graph 1152. Functional ultrasound imaging (fUSI) visualizes neural activity by mapping local changes in cerebral blood volume (CBV). CBV variations are tightly linked to neuronal activity through the neurovascular coupling and are evaluated by calculated power Doppler variations in the brain. In the tests described below, the fUSI system used an ultrasonic probe centered at 7.5 MHZ (Bandwidth >60%, 128 elements, 0.300 mm pitch, from Vermon of Tours, France) connected to a Verasonics Vantage ultrasound system (Verasonics Inc. of Redmond, WA, USA) controlled by custom MATLAB (MathWorks, USA) B-mode and fUSI acquisition scripts. Each power Doppler image was obtained from the accumulation of 300 compounded frames acquired at 400 Hz frame rate. Each compounded frame was created using 2 accumulations of 5 tilted plane waves (−6°,−3°, 0°, 3°, 6°). A pulse repetition frequency (PRF) of 4000 Hz was used. fUSI images were repeated every 1.65 seconds. Each block of 300 images was processed using a SVD clutter filter to separate tissue signal from blood signal as shown in a graph 1154 to obtain a final power Doppler image 1156 through the scalp of a patient having the example PMMA cranial implant exhibiting artificial (for in vitro experiment) or cerebral blood volume (CBV) in the whole imaging plane.

The example implant 1110 was tested using the fUSI based process in FIG. 11E to acquire a series of sequential power Doppler images and observing spatiotemporal changes in signals. FIG. 12 shows a phantom test system 1200 to determine if power Doppler signals could be detected through the PMMA material for the implant 1110. The test system 1200 includes a box frame 1210 with two parallel structures 1212 and 1214 to create a scaffold for supporting polyethylene tubes. The box fame 1210 is hollow, box-shaped, 3D-printed, and nylon cast. A set of 280-μm inner diameter polyethylene tubes 1220 are routed through the frame 1210 at three lateral positions and five axial positions (15 grid points, total) between the structures 1212 and 1214. A liquid gelatin phantom 1222 with graphite added was poured around the tubing 1220 to mimic the scattering effects of biological soft tissue. Once the phantom cast 1222 had set/solidified, a red blood cell phantom liquid (CAE Blue Phantom™ Doppler Fluid) was flowed through the tubing 1220 using a peristaltic pump and a long recirculating route with a low pass filter to create a smooth flow at velocities of approximately 0.1 mL/min. The phantom system 1200 is a Doppler ultrasound phantom with flow channels from the tubing 1220 designed to mimic blood flow in a human brain. A test layer 1230 representing different thickness implants is imposed over the imaging plane. An ultrasound transducer probe 1232 was inserted on the test layer 1230 to capture power Doppler images. This allowed measurement of the signals underlying power Doppler images in a controlled environment.

Five different imaging scenarios were compared using the test system 1200. The five scenarios included: (1) no implant, (2) a 1 mm thick PMMA implant, (3) a 2 mm thick PMMA implant, (4) a 3 mm thick PMMA implant, and (5) a titanium mesh implant. The titanium mesh implant was fabricated from pure titanium of 0.6 mm thickness with honeycomb patterns alternating between small circles (1.5 mm diameter) and big circles (3 mm diameter). Synthetic red blood cells were passed through the 280-μm diameter tubing 1220 at three lateral (5, 15, 25 mm) and four axial positions (14, 24, 34, 44 mm) at a constant velocity of ˜27 mm/s. The red blood cells were imaged with a linear ultrasound array transmitting at 7.5 MHz, and power Doppler signals were recorded to estimate the signal-to-noise ratio (SNR) and resolution loss in each imaging scenario.

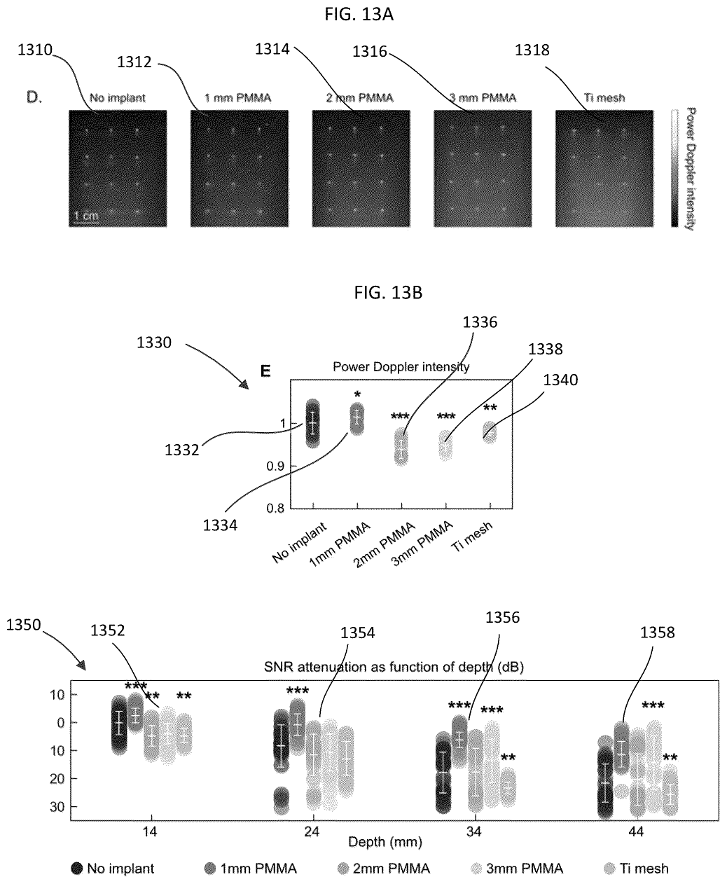

FIG. 13A shows a series of power Doppler images 1310, 1312, 1314, 1316, and 1318 of the Doppler blood flow phantom in the test system 1200 of FIG. 12 with different implant scenarios. The scales indicates relative power Doppler signal intensity. The image 1310 was taken with no implant. The image 1312 was taken with the 1 mm thick implant, the image 1314 was taken with the 2 mm thick implant, and the image 1316 was taken with the 3 mm thick implant. The image 1318 was taken with the titanium mesh implant.

FIG. 13B shows a graph 1330 plotting power Doppler intensity for each implant over N=15 acquisitions. A bar 1332 represents the intensity with no implant, a bar 1334 represents the intensity with the 1 mm thick implant, a bar 1336 represents the intensity with the 2 mm thick implant, and a bar 1338 represents the intensity with the 3 mm thick implant, and a bar 1340 represents the intensity with the titanium mesh. Stars indicate statistical differences (paired sampled t-test) between each scenario compared to the no implant scenario (*: p<0.05, **: p<0.01, ***: p<0.001). Data are presented as a mean±standard deviation (represented by the respective horizontal and vertical lines in the bars of dots 1332, 1334, 1336, 1338, and 1340, dots in the bars 1332, 1334, 1336, 1338, and 1340 represent individual acquisitions, n=15 acquisitions per group.

FIG. 13B shows a graph 1350 plotting signal noise ratio (SNR) attenuation for each implant scenario as respective groups of bars 1352, 1354, 1356, and 1358, each with five bars representing no implant, the 1 mm thick implant, the 2 mm thick implant, the 3 mm thick implant, and the titanium mesh respectively. The groups of bars 1352, 1354, 1356, and 1358 are plotted as a function of the depth (N=15 acquisitions) for the depths of 14, 24, 34, and 44 mm respectively. All values in the graph 1350 were normalized with the mean value of the no implant control. Dots represent individual acquisitions. The vertical lines represent standard deviation while the horizontal lines represent mean value within each bar.

It was found that signal intensity decreased with increasing PMMA implant thickness, and that image quality was most strongly degraded by the titanium mesh as shown in the image 1318 in FIG. 13A and the graph of Doppler intensity 1330 in FIG. 13B. The SNR decreased with depth and was inversely proportional to the thickness of the intervening PMMA material as shown in the graph 1350 in FIG. 13B.

To test the ability to detect functional brain signals through the different cranial implant materials in vivo, fUSI was performed in four rats after placing each of the implant types on top of their brain following an acute craniotomy. As the fUSI signals were recorded, a passive visual simulation task designed to activate the visual system of the test rats was used.

Four Long-Evans male rats were used in the test (15-20 weeks old, 500-650 g). During the surgery and the subsequent imaging session, the test rats were anesthetized using an initial intraperitoneal injection of xylazine (10 mg/kg) and ketamine (Imalgene, 80 mg/kg). The scalps of the test rats were removed, and the skulls were cleaned with saline. A craniectomy was performed to remove 0.5 mm×1 cm of the skull by drilling (Foredom) at low speed using a micro drill steel burr (Burr number 19007-07, Fine Science Tools). After surgery, the surface of the brain was rinsed with sterile saline and ultrasound coupling gel was placed in the window. The linear ultrasound transducer was positioned directly above the cranial window and a fUSI scan was performed. The 1 mm, 2 mm, and 3 mm thick PMMA materials or the titanium mesh, were placed above the brain, and the fUSI acquisition was repeated.

To quantitatively characterize the fUSI sensitivity through the different PMMA thicknesses, the SNR of blood vessels in the cortex and in deeper thalamic regions from the same animal with different implants was calculated. A first region of interest (ROI) of the cortex and a second ROI of the deeper regions was selected for each implant condition. For each horizontal line of these ROIs, the lateral intensity was plotted, and local maxima (blood vessels) and minima were identified. The SNR was then calculated as: SNR=(local maxima)/(local minima).

fUSI with visual stimuli was performed in one of the test rats. Visual stimuli were delivered using a blue LED (450 nm wavelength) positioned at 5 cm in front of the head of the rat. Stimulation runs consisted of periodic flickering of the blue LED (flickering rate: 5 Hz) using the following parameters: 50 seconds dark, followed by 16.5 seconds of light flickering repeated three times for a total duration of 180 seconds. At this distance, the light luminance was of 14 lux when the light was on and ˜0.01 lux when the light was off.

For the rodent and human in vivo experiments, a General Linear Model (GLM) was used to find which voxels were significantly modulated by the visual task. To perform this GLM, the fUSI data was preprocessed with nonlinear motion correction, followed by spatial smoothing (2D Gaussian with sigma=1 (FWHM=471 μm), followed by a voxelwise moving average temporal filter (rat: 2-timepoints; human: 5-timepoints). The fUSI signal was scaled by its voxelwise mean so that all the runs and voxels had a similar signal range. To generate the GLM regressor for the visual task, the block task design was convolved with a single Gamma hemodynamic response function (HRF). For the rodent experiments, the HRF time constant was (τ)=0.7, time delay (δ)=1 sec, and phase delay (n)=3 sec. For the human experiments, the values were τ=0.7, δ=3 sec, n=3 sec. The GLM was fit using the convolved regressor and the scaled fUSI signal from each voxel. The statistical significance of the beta coefficients was determined for each voxel using a 2-sided t-test with False Discovery Rate (FDR) correction (p (corrected)<10⁻⁵).

The test rats experienced subsequent blocks of darkness (50 seconds) and light exposure (16.5 seconds). The response of each voxel to the visual stimulation was modeled using the general linear model (GLM), which allowed quantification of which voxels showed significant visual modulation (p<10⁻⁵). Briefly, the block design (“rest” or “light”) was convolved with the hemodynamic response function and linear model mapping of the convolved stimulation regressors was fit to each fUSI voxel's signal. This allowed assessment of the statistical significance between the hemodynamic response and the stimulation structure for each voxel.

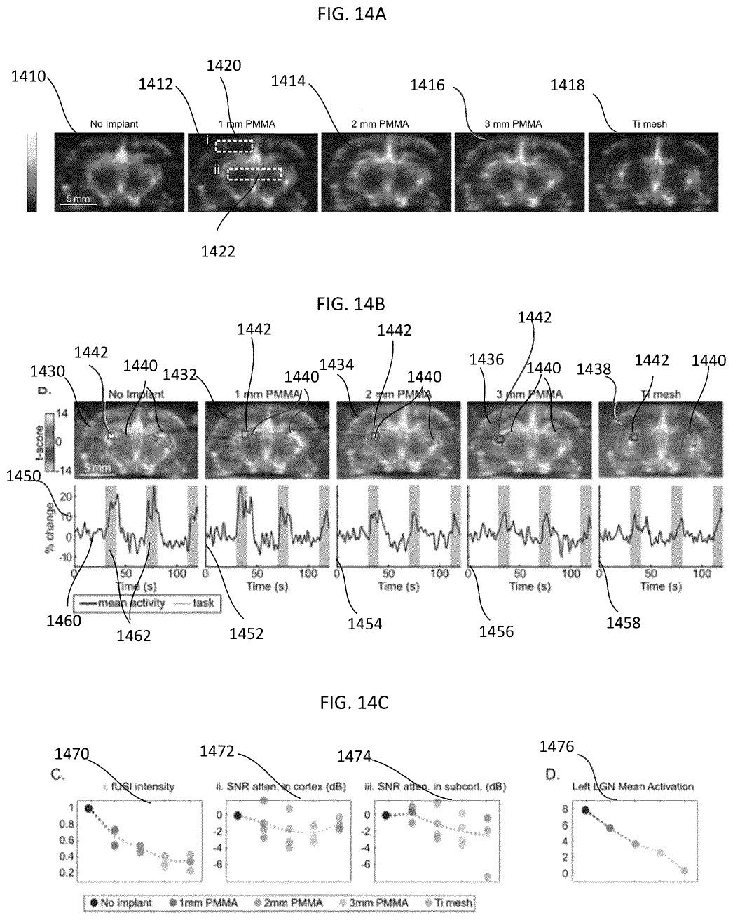

FIG. 14A shows in vivo fUSI images 1410, 1412, 1414, 1416, and 1418 of coronal plane of the same rat brain with the different skull implants. The image 1410 is taken with no implant, the image 1412 is taken with a 1 mm thick implant, the image 1414 is taken with a 2 mm thick implant, the image 1416 is taken with a 3 mm thick implant, and the image 1418 is taken with a titanium mesh. The scale in the images 1410, 1412, 1414, 1416, and 1418 indicates fUSI signal intensity. A box 1420 and a box 1422 in the image 1412 correspond to studied cortical and sub-cortical regions respectively.

FIG. 14B shows fUSI images 1430, 1432, 1434, 1436, and 1438 of the rat brain with no implant and the implant types explained above respectively in functional response to visual stimuli in highlighted areas 1440. Each of the images 1430, 1432, 1434, 1436, and 1438 have a background image that is the functional ultrasound image with areas 1440 of significant response highlighted. All voxels with significant response to visual stimulation (p corrected<10⁻⁵) have T-score overlaid as indicated in the areas 1440. A general linear model (GLM) was used to calculate the T-scores and significances. Boxes 1442 in the images 1430, 1432, 1434, 1436, and 1438 show the lateral geniculate nucleus (LGN) region used to calculate mean fUSI over time.

FIG. 14B also shows graphs 1450, 1452, 1454, 1456, and 1458 of time course from the LGN region for each skull implant condition (no implant, 1 mm, 2 mm, 3 mm, and titanium mesh respectively). In the graphs 1450, 1452, 1454, 1456, and 1458, a plot 1460 represents the mean percent change, and bars 1462 represent the two light-on conditions to the test rats.

FIG. 14C shows a graph 1470 of standardized fUSI intensity for scans for each of the test rats with no implant, the 1 mm implant, the 2 mm implant, the 3 mm implant, and the titanium mesh. FIG. 14C shows a graph 1472 of standardized SNR in the cortex (corresponding to the region of the box 1420 in the image 1412 in FIG. 14A for the cortex region) and a graph 1474 of standardized SNR in subcortical structures (correspond to the region of the box 1420 in the image 1412 in FIG. 14A of the subcortical structures region) for scans for each of the test rats with no implant, the 1 mm implant, the 2 mm implant, the 3 mm implant, and the titanium mesh. All values were normalized with the mean value of the no implant control. Statistical significance between standardized SNR for no implant and each skull implant scenario were non-significant. A graph 1476 shows a significant activated area of the left LGN (in mm²) following visual stimulation as a function of no implant, the 1 mm implant, the 2 mm implant, the 3 mm implant, and the titanium mesh (P<0.05). The data in the graphs 1470, 1472, 1474, and 1476 were analyzed by 1-way ANOVA with post-hoc Tukey hsd for significant differences between each implant compared to the no implant control (* p<0.05, ** p<0.01, *** p<0.001).

The in vivo performance of fUSI through the implants were numerically evaluated by first calculating the overall fUSI signal intensity received through the different PMAA thicknesses and through the titanium mesh. As shown in the graph 1470, the total fUSI intensity from the whole brain decreased by 30% from the no implant scenario to the 1 mm implant. The fUSI intensity dropped a further ˜15% per mm implant thickness for the 2 mm and 3 mm materials. The fUSI intensity decreased by 60% for the titanium mesh compared to no implant. The SNR of the cerebral blood vessels captured with the fUSI sequence were calculated. The graphs 1472 and 1474 in FIG. 14C show that the SNR decreased as the PMMA implant thickness increased but with a smaller decrease than observed for fUSI intensity. In the cortex, the SNR decreased slightly with the mesh (−1 dB) and as the PMMA implant thickened (˜−1 dB/mm). The subcortical structures within the image showed a similar trend in SNR across the different implant materials.

In all five implant conditions, voxels within the lateral geniculate nucleus (LGN) activated during optical stimulation were identified as shown in the images 1430, 1432, 1434, 1436, and 1438 in FIG. 14B. Using fUSI through the thicker implants and the titanium mesh resulted in fewer activated voxels within the LGN. Additionally, less signal change was observed for the thicker implants in relation to the thinner implants and the titanium mesh in comparison to any of the PMMA implants (p<10⁻³, 1-way ANOVA with post hoc hsd shown in the graph 1476 in FIG. 14C). Taken together, the in vitro and in vivo results show that PMMA is superior to the titanium mesh as an intervening material for fUSI and that making the PMMA window as thin as possible offers the best imaging performance.

To test the possibility of performing fUSI through a chronic cranial window, a human participant that was an adult male in his thirties was tested. Approximately 30 months prior to skull reconstruction, the human participant suffered a traumatic brain injury and underwent a left decompressive hemicraniectomy of approximately 16 cm length by 10 cm height.

Anatomical and functional MRI scans allowed mapping of brain structures and functional cortical regions within the borders of the craniectomy. The test participant underwent an fMRI scan during which he performed a finger tapping task with a block design of 30 second rest followed by 30 second sequential finger tapping with his right hand. These blocks were repeated 7 times for a total scan duration of 8 minutes. Instructions for start and end of finger tapping epochs were delivered with auditory commands delivered through MR compatible headphones. The fMRI acquisition was done on a 7T Siemens Magnetom Terra system with a 32-channel receive 1Tx head coil with a multi-band gradient echo planar imaging (GE-EPI) T2*-weighted sequence with 1 mm³ isotropic resolution, 192 mm×192 mm FOV, 92 axial slices, TR/TE 3000 ms/22 ms, 160 volumes, FA 80 deg, A-P phase encoding direction, iPAT=3 and SMS=2. An anatomical scan was also acquired using a T1-weighted MPRAGE sequence with 0.7-mm³ isotropic resolution, 224 mm×224 mm FOV, 240 sagittal slices, TR/TE 2200 ms/2.95 ms, FA 7-deg. Statistical analysis of fMRI data was performed with a GLM using Statistical Parametric Mapping (SPM12). Preprocessing included motion-realignment, linear drift removal, and co-registration of fMRI data to a high-resolution anatomical scan.

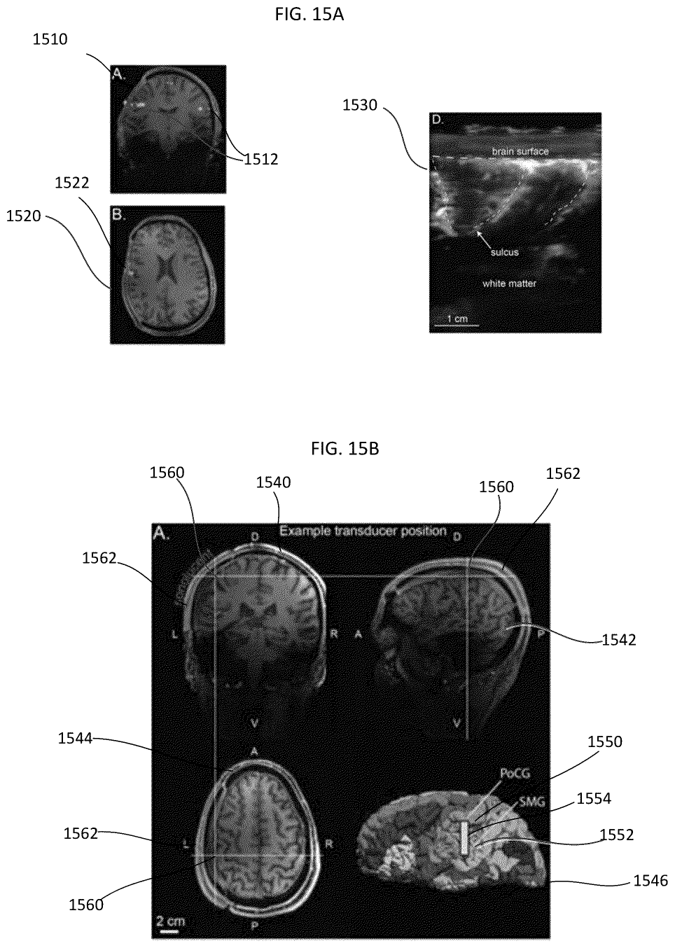

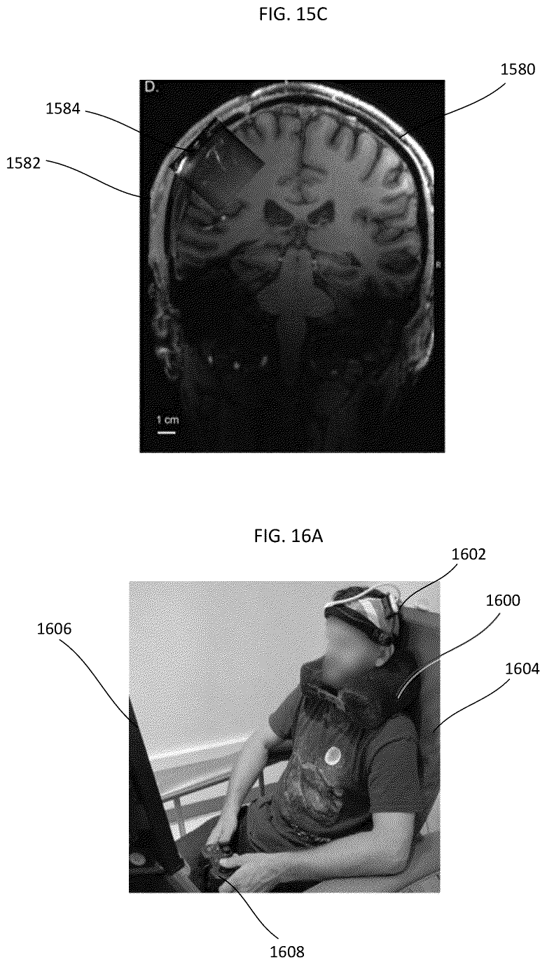

FIG. 15A shows a screen shot 1510 of a fMRI showing a coronal plane of the test participant after the decompressive hemicranicctomy. An overlay shows regions 1512 activated during the finger tapping task. A screen shot 1520 of a fMRI of the transverse plane of the test participant shows finger-tapping regions 1522 of the test participant. Before reconstruction, the brain of the test participant was imaged participant through the scalp using power Doppler ultrasound through the intact scalp, with no intervening bone as shown in an image 1530. Large brain vessels were observed following the curve of sulci folds and smaller vessels irrigating the sulci, typical of fUSI images. However, due to the lack of intracranial pressure and the dramatic brain motion that results from this condition, functional data or co-registration of ultrasound images to anatomical MRIs was not possible. Nevertheless, the ability to collect high quality vascular maps provided evidence that fUSI was possible through an intact human scalp.

To successfully detect a functional signal through a customized example cranial implant (CCI) for the test participant, an appropriate acoustic window was designed with the implant according to the principles described herein. In the separate fMRI study, cortical response fields to a simple finger tapping task were identified prior to his skull reconstruction via the fMRI in the screen shots 1510 and 1520. In this example, the fMRI scans indicated the locations of the “finger tapping” regions of the cortex as shown in regions 1522. The implant was then designed so that the thinned portion lay over this area of cortex. Based on this mapping, the example PMMA CCI implant was designed with a 2 mm thick, 34×50 mm parallelogram-shaped sonolucent “window to the brain.” The 2 mm-thick portion window in this example was designed to sit above the primary motor cortex, primary somatosensory cortex, and posterior parietal cortex of the brain for fUSI acquisition from these areas. Alternatively, the window could be designed to sit above one or two of these areas in the brain or other areas of interest of the brain. The remaining sections of the PMMA implant were 4 mm thick. This implant design was calculated by the manufacturer of a standard skull implant such as Longeviti ClearFit to provide sufficient mechanical performance to serve as a permanent skull replacement.

The testing involved reconstructing the skull of the test participant with the example customized implant with the acoustic window as shown in FIG. 11B. FIG. 15B shows fMRI scans of the brain of the test participant after the reconstruction. A first image 1540 shows a scan of a coronal view of the brain at approximately the level of the parietal cortex. A second image 1542 shows a scan of a side view of the brain. A third image 1544 shows a scan of a horizontal slice of the brain. In the images 1540, 1542, and 1544, D stands for dorsal; V stands for ventral, A stands for anterior; P stands for posterior, L stands for left; and R stands for right.

A brain schematic 1546 was generated via 3D reconstruction in BrainSuite, software provided by UCLA, combined with 3D modeling in SolidWorks. The brain schematic 1546 includes an area 1550 that is the Postcentral gyrus (poCG) and an area 1552 that is the Supramarginal gyrus (SMG). A bar 1554 in the brain schematic 1546 is the estimated position of the ultrasound transducer.

A set of white crosses 1560 in the images 1540, 1542, and 1544 indicate the middle of the transducer during the example fUSI session. An area 1562 indicates the sonolucent portion of the head including the scalp, the customized cranial implant, and the meninges above the brain. FIG. 15C shows an image 1580 that is co-registration of the fUSI imaging plane with an anatomical magnetic resonance image. The image 1580 shows the implant area 1582 and superimposed fUSI image 1584. The fUSI image 1584 and anatomical MRI were manually co-registered and represents the best prediction of the observed fUSI 2D plane location.

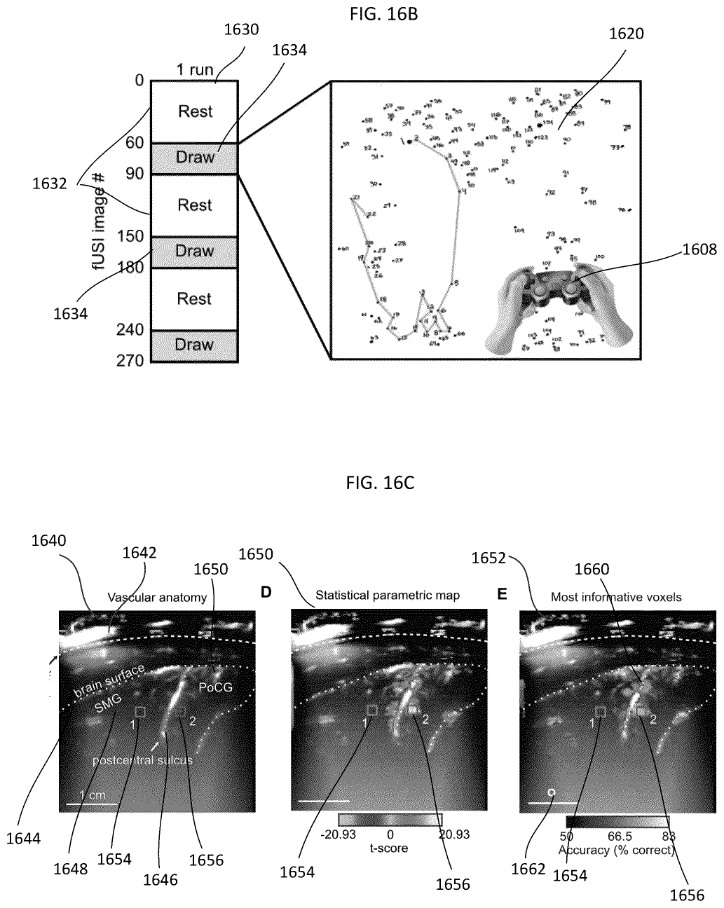

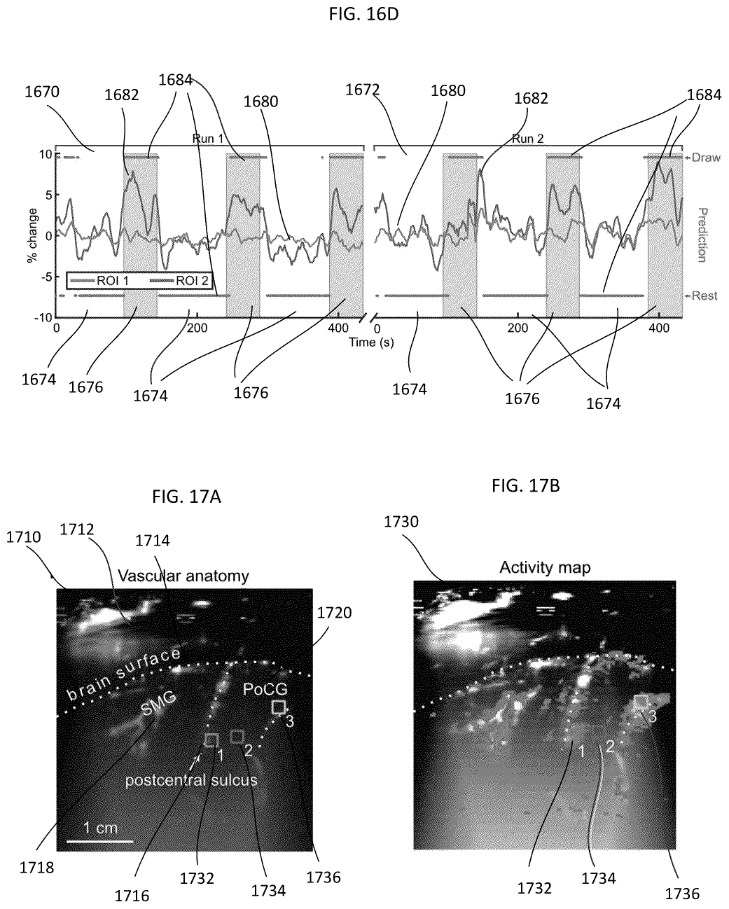

The testing showed that the acoustic window in the example customized implant allowed fUSI activity recording in the fully reconstructed human participant. The tests showed that ultrasound enables vascular imaging through the intact scalp after decompressive craniectomy. The brain of the test participant was imaged through the acoustic window following the skull reconstruction. The boundaries of the thinned window in the implant were located using real-time anatomical B-mode ultrasound imaging. Once the boundaries of the “window” were located, a custom-designed cap was used to position and stabilize the ultrasound transducer above the middle of the acoustic 2 mm thick window as shown in FIG. 11B. The cortical vasculature, including vessels following the curves of sulcal folds and smaller vessels irrigating the adjacent cortex were immediately observed in a power Doppler image 1640 shown in FIG. 16C.

Based on a prior fUSI recording session and the location of the thinned window, it was estimated the transducer was positioned above the left primary somatosensory cortex (S1) and supramarginal gyrus (SMG), with the S1 playing a role in processing somatic sensory signals from the body and the SMG playing a role in grasping and tool use. Thus, to detect functional brain signals after the reconstruction of the cranial implant, the test participant was instructed to perform two visuomotor tasks. Ten to twelve months after skull reconstruction, the test participant underwent fUSI scans during performance of the visuomotor tasks.

FIG. 16A shows the testing environment for fUSI scans during performance of the two visuomotor tasks. The test participant 1600 with the example customized implant had a fUSI transducer probe 1602 attached. The test participant 1600 was seated in a reclining chair 1604 with a 27-inch fronto-parallel screen 1606 (Acer XB271HU) positioned 70 cm in front of him. The participant controlled the first behavioral task using a Logitech F310 Gamepad 1608. Gopher software (https://github.com/Tylemagnc/Gopher360) was used to enable control of the computer with the Logitech Gamepad 1608. In the first task, the right thumbstick controlled the position of the computer cursor on the screen 1606, whereas the left shoulder button functioned as the left mouse button. A block design for the drawing task was used with 100-second rest blocks followed by 50 seconds of drawing with the Gamepad 1608. The participant was verbally instructed for each rest or task block. The participant was instructed to complete one of multiple “Connect-The-Dots” drawings such as the drawing 1620 shown in FIG. 16B. When the test participant finished one of the drawings, a new drawing was presented for him to complete. For the rest blocks, the test participant was instructed to rest with closed eyes and relaxed mind. The fUSI data was acquired at 0.6 Hz (1.65 sec/frame).

The second visuomotor task was a guitar playing task. The test environment was used with the test participant playing a guitar in place of operating the game controller. An identical block design with 60-frame rest blocks followed by 30 frame task blocks was used. In the task blocks, the test participant used the left hand to form chords on the fretboard and the right hand to strum the strings.

To decode whether a given timepoint was in a “task” or “rest” block, a principal component analysis (PCA) was used for dimensionality reduction and linear discriminant analysis (LDA) for classification. Each motion-corrected fUSI timepoint (“sample”) was labeled as “rest” or “task.” The dataset was balanced to have an equal number of “rest” and “task” timepoints. The dataset was then split into block pairs (1 block pair=rest+task) to avoid training the classifier on time points immediately adjacent to the test time points. This helped ensure that the model would generalize and that the model was not memorizing local patterns for each block pair. A 2D Gaussian smoothing filter (sigma=1) was applied to each sample in the training and test sets. The training set was z-scored across time for each voxel. The PCA+LDA classifier was then trained and validated using a block-wise leave-one-out cross-validator. 5 blocks were used for training and then tested on the held-out block pair's timepoints. For the PCA, 95% of the variance was kept. To generate the example session decoding, 5 blocks were used for training with balanced samples of “rest” and “draw” and then tested on the unbalanced final block (60 fUSI frames of rest data and 30 fUSI frames of draw task).

Searchlight analysis was used to identify how much task information different parts of an image or volume contain. The searchlight analysis produced information maps by measuring the decoding performance in small windows, or “searchlights”, centered on each voxel. For the searchlight analysis, a circular region of interest (ROI) of 600 μm radius was used and the task decoding analysis was performed using only the pixels within the ROI. The percent correct metric of the ROI was assigned to the center voxel. This was repeated across the entire image, such that each image pixel is the center of one ROI. To visualize the results, the percent correct metric was overlaid onto a vascular map and the 5% most significant voxels were kept. The searchlight analysis was run only on brain voxels, ignoring all voxels above the brain surface.

Unless otherwise stated, a significant difference was considered P<0.01. Comparisons between two groups were performed using a two-sided t-test. Comparisons between more than two groups were performed using 1-way ANOVA with Tukey hsd post-hoc test. For the decoding analysis, a binomial test was used to assess statistical significance (P<10⁻¹⁰). All statistical analysis was performed in MATLAB 2021b. For the GLM, P-values were corrected for multiple testing using the False Discovery Rate method. For the comparison between SNR of skull implant conditions, P-values were corrected for multiple testing using the Bonferroni method.

FIG. 16B also shows a timeline 1630 of fUSI images collected over rest blocks 1632 and draw blocks 1634 for the connect-the-dot video gaming task. In the rest blocks, the participant relaxed and tried to keep a clear mind. In the task blocks, the test participant used the gamepad 1608 in FIG. 16A to draw lines in a “connect-the-dots” task as shown in an example image 1620.

FIG. 16C shows a power Doppler image 1640 of the vascular anatomy of the imaging plane of the test participant. Areas defined by dashed lines highlight specific anatomic features as labeled, including a PMMA implant surface 1642, a brain surface 1644, and sulcal vessels 1646. Another area 1648 represents the Supramarginal gyrus and an area 1650 represents the Postcentral gyrus. A statistical parametric map 1650 represents areas across two concatenated runs and is a T-score statistical parametric map, where values shown for voxels where P_{corrected}<10⁻¹⁰. An image 1652 shows a mapping of the searchlight of the most informative voxels. Boxes 1654 and 1656 show respective first and second regions of interest (ROI 1 and ROI 2) used in the power Doppler image 1640, the statistical parametric map 1650, and the searchlight mapping 1652.

The mapping 1652 is a searchlight analysis showing which small subsets of image voxels contain the most task information. A background image is a functional ultrasound image. The top 5% of searchlight windows with the highest decoding accuracy are superimposed as voxels 1660. The superimposed voxels 1660 correspond to a significance threshold of Pcorrected p(corrected)<2.8×10⁻⁴. A circle 1662 represents a 600 μm searchlight radius.

FIG. 16D shows a graph 1670 of mean scaled fUSI signals from the first and second region of interest during a first run and a graph 1672 of mean scaled fUSI signals from the first and second region of interest during a second run. Due to the density of these points, the red circles overlap and appear as a solid line. The graphs 1670 and 1672 shows a set of regions 1674 that are rest blocks, and a set of regions 1676 that are task blocks. A plot 1680 represents the mean scaled fUSI signals in the first region of interest. A plot 1682 represents the mean scaled fUSI signals in the second region of interest. A set of circles 1684 show predictions from the linear decoder for rest and draw periods.

In the first task, a block design with 100-second “rest” blocks and 50-second “task” block was used. During the rest blocks, the participant was instructed to close his eyes and relax. During the task blocks, the participant used the video game controller joystick to complete “connect-the-dots” puzzles on the display 1606 in FIG. 16A. The same tasks were repeated across multiple runs (N=3). Finally, the data from two runs was concatenated and a GLM analysis was used to identify voxels with functional activation. The GLM revealed several regions that were task-modulated as shown in the T-score parametric map 1650 in FIG. 16C. The activity within these regions displayed positive modulation by the task, i.e., increased activity during the drawing blocks and decreased activity during the rest blocks as shown in the second region of interest in the graph 1670 and 1672 in FIG. 16D. For example, ROI 2 had an average of 3.68% difference between the drawing and rest blocks (p value <10⁻¹⁰, two-sided t-test). Outside of these activated regions (i.e., ROI 1 in the graphs 1670 and 1672), the signal remained stable throughout the run with no significant increase nor decrease during the task periods. For example, ROI 1 had an average difference of −0.034% between the drawing and rest blocks, (p value=0.67, two-sided t-test).

As a first step towards human BMI applications, the ability to decode task state (rest vs. connect-the-dot) was tested from single trials of the fUSI data using a linear decoder. The task state was decided with 84.7% accuracy (p<10⁻¹⁵, 1-sided binomial test). To better understand which voxels in the image contained the most information discriminating the task blocks, a searchlight analysis with a 600 μm radius was performed as shown in the searchlight map 1652 in FIG. 16C. This analysis showed that the 5% most informative voxels were distributed across the image and closely matched the results of the statistical parametric map from the GLM. When the decoder accuracy across the example session was examined, the linear decoder predicted both the draw and rest blocks with similarly high accuracy as shown in the graphs 1670 and 1672 in FIG. 16D, with most of the errors occurring at the transitions between the two task states. This effect is likely due in part to the latency between the neural activity and resulting hemodynamic response.

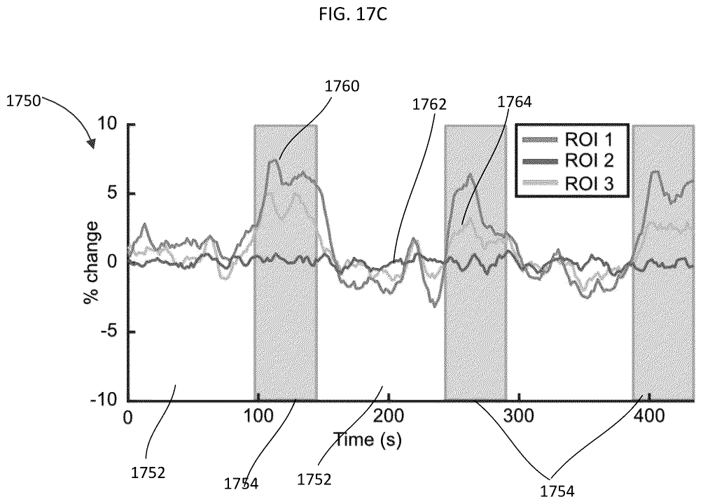

In the second task in the testing, the test participant played guitar while fUSI data was recorded. FIG. 17A shows a power Doppler image 1710 showing the vascular anatomy of the imaging plane of the test participant. Areas defined by dashed lines highlight specific anatomic features as labeled, including a PMMA implant surface 1712, a brain surface 1714, and sulcal vessels 1716. Another area 1718 represents the Supramarginal gyrus and an area 1720 represents the Postcentral gyrus. FIG. 17B shows a T-score statistical parametric map 1730 of activity in the guitar playing task, thresholded at P_{corrected}<10⁻¹⁰. A set of boxes 1732, 1734, and 1736 show respective regions of interest in both images 1710 and 130.

FIG. 17C shows a graph 1750 plotting the mean scaled fUSI signal from the ROIs during guitar-playing task. The graph 1750 shows a set of regions 1752 that are rest blocks, and a set of regions 1754 that are task blocks. A plot 1760 represents the mean scaled fUSI signals in the first region of interest. A plot 1762 represents the mean scaled fUSI signals in the second region of interest. A plot 1764 represents the mean scaled fUSI signals in the third region of interest.

During the rest blocks (100-second), the test participant was instructed to minimize finger/hand movements, close his eyes, and relax. During the task blocks (50 seconds), the participant played improvised or memorized music on a guitar with his right-hand strumming and his left fingers moving on the fretboard. Several regions that were task-activated were identified, including several that were similar in location to those activated by the connect-the-dots task as shown in FIGS. 17B-17C.

The example cranial implant having a sonolucent window may be used for clinical use for diagnostics analysis and monitoring after skull reconstruction (clinical use). fUSI systems and the example customized cranial implant (CCI) with an acoustic window allows routine monitoring during the postoperative period for both anatomical and functional recovery. In addition to generalized post-operative monitoring, some TBI patients will develop specific pathologies that would benefit from increased monitoring frequency. For example, Syndrome of the Trephined (SoT) is an indication where patients develop neurological symptoms such as headaches, dizziness, and cognitive impairments due to altered cerebrospinal fluid dynamics and changes in intracranial pressure following a large craniectomy. Recording from these patients with TBI sequelae or SoT may provide novel insight into the pathophysiology of their disease processes and subsequent recovery.

Another application of the example interface may be for research in Neuroscience and brain-machine interfaces. If only a small fraction of these patients receives a cranial implant with an acoustic window as part of their standard of care, it would provide a major opportunity to measure mesoscopic neural activity with excellent spatiotemporal resolution and high sensitivity in humans. In those patients with minimal long-term neurological damage, it will also enable new investigations into advanced neuroimaging techniques and BMI. fUSI possesses high sensitivity even through the acoustic window. Not only can task-modulated areas be identified by averaging across all task blocks and using a GLM resulting in a statistical parametric map such as the map 1650 in FIG. 16C, but a linear decoder may robustly decode the current task block using single fUSI images such as those shown in the mapping 1652 and the graph 1670 in FIGS. 16B and 16D.

The example implant may also be applied to any optical or acoustic technique to image or otherwise measure anatomical or functional brain signals through a translucent and/or sonolucent (acoustically transparent) skull replacement materials, including but not limited to titanium mesh and PEEK. Use of the example interface may also be extended to decoding non-motor information from the brain, including but not limited to sensory information, for neuropsychiatric and cognitive disorders.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In one or more embodiments, computer-executable instructions are executed on a general purpose computer to turn the general purpose computer into a special purpose computer implementing elements of the disclosure. The computer executable instructions may be, for example, binaries, intermediate format instructions such as assembly language, or even source code. Although the subject matter has been described in language specific to structural marketing features and/or methodological acts, it is to be understood that the subject matter defined in the appended claims is not necessarily limited to the described marketing features or acts described above. Rather, the described marketing features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as an un-subscription model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing un-subscription model can be composed of various characteristics such as, for example, on-demand self-service, broad network access, resource pooling, rapid elasticity, measured service, and so forth. A cloud-computing un-subscription model can also expose various service un-subscription models, such as, for example, Software as a Service (“SaaS”), a web service, Platform as a Service (“PaaS”), and Infrastructure as a Service (“IaaS”). A cloud-computing un-subscription model can also be deployed using different deployment un-subscription models such as private cloud, community cloud, public cloud, hybrid cloud, and so forth. In this description and in the claims, a “cloud-computing environment” is an environment in which cloud computing is employed.

In one example, a computing device may be configured to perform one or more of the processes described above. the computing device can comprise a processor, a memory, a storage device, an I/O interface, and a communication interface, which may be communicatively coupled by way of a communication infrastructure. In certain embodiments, the computing device can include fewer or more components than those described above.

In one or more embodiments, the processor includes hardware for executing instructions, such as those making up a computer program. As an example and not by way of limitation, to execute instructions for digitizing real-world objects, the processor may retrieve (or fetch) the instructions from an internal register, an internal cache, the memory, or the storage device and decode and execute them. The memory may be a volatile or non-volatile memory used for storing data, metadata, and programs for execution by the processor(s). The storage device includes storage, such as a hard disk, flash disk drive, or other digital storage device, for storing data or instructions related to object digitizing processes (e.g., digital scans, digital models).

The I/O interface allows a user to provide input to, receive output from, and otherwise transfer data to and receive data from computing device. The I/O interface may include a mouse, a keypad or a keyboard, a touch screen, a camera, an optical scanner, network interface, modem, other known I/O devices or a combination of such I/O interfaces. The I/O interface may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface is configured to provide graphical data to a display for presentation to a user. The graphical data may be representative of one or more graphical user interfaces and/or any other graphical content as may serve a particular implementation.

The communication interface can include hardware, software, or both. In any event, the communication interface can provide one or more interfaces for communication (such as, for example, packet-based communication) between the computing device and one or more other computing devices or networks. As an example and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI.

Additionally, the communication interface may facilitate communications with various types of wired or wireless networks. The communication interface may also facilitate communications using various communication protocols. The communication infrastructure may also include hardware, software, or both that couples components of the computing device to each other. For example, the communication interface may use one or more networks and/or protocols to enable a plurality of computing devices connected by a particular infrastructure to communicate with each other to perform one or more aspects of the digitizing processes described herein. To illustrate, the image compression process can allow a plurality of devices (e.g., server devices for performing image processing tasks of a large number of images) to exchange information using various communication networks and protocols for exchanging information about a selected workflow and image data for a plurality of images.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above-described embodiments. Rather, the scope of the invention should be defined in accordance with the following claims and their equivalents.

1. A cranial implant replacing a section of a skull for functional ultrasound imaging, the cranial implant comprising:
a support section shaped to replace a removed section of the skull; and
a window of sonolucent material in the support section allowing functional ultrasound imaging through an ultrasound probe on the window, wherein the window is shaped to allow access to a region of interest in the brain.

2. The cranial implant of claim 1, wherein the window of the sonolucent material includes the entire support section.

3. The cranial implant of claim 1, wherein the sonolucent window is fabricated from one of polymethyl methacrylate (PMMA) or polyether ether ketone (PEEK).

4. The cranial implant of claim 1, wherein the sonolucent window is a titanium mesh.

5. The cranial implant of claim 1, wherein the sonolucent window is positioned above at least one of a primary motor cortex, a primary somatosensory cortex, or a posterior parietal cortex of the brain when the support section replaces the removed section of the skull.

6. The cranial implant of claim 1, wherein the sonolucent window has a thickness of between 1 and 10 mm.

7. The cranial implant of claim 6, wherein the sonolucent window is a subsection of the support section, and wherein the sonolucent window has a thickness less than the thickness of the support section.

8. The cranial implant of claim 1, wherein the ultrasound probe is positioned near a Postcentral gyrus (poCG) and a Supramarginal gyrus (SMG) of the brain.

9. The cranial implant of claim 1, wherein the window is a parallelogram shape.

10. A method for producing a customized cranial implant for collecting functional ultrasound images, the method comprising:
performing a craniectomy on a patient;
imaging the brain underneath a skull section removed through the craniotomy;
fabricating a cranial implant to replace the skull section removed through the craniotomy;
creating a sonolucent window in the implant above a region of the brain based on the imaging to allow collection of functional ultrasound images via an ultrasound transducer probe.

11. The method of claim 10, wherein the imaging is performed via either a magnetic resonance imaging system or a functional ultrasound system.

12. The method of claim 10, wherein the window of the sonolucent material includes the entire support section.

13. The method of claim 10, wherein the sonolucent window is fabricated from one of polymethyl methacrylate (PMMA) or polyether ether ketone (PEEK).

14. The method of claim 10, wherein the sonolucent window is a titanium mesh.

15. The method of claim 10, wherein the sonolucent window is positioned above at least one of a primary motor cortex, a primary somatosensory cortex, or a posterior parietal cortex of the brain.

16. The method of claim 10, wherein the sonolucent window has a thickness of between 1 and 10 mm.

17. The method of claim 16, wherein the sonolucent window is a subsection of the support section, and wherein the sonolucent window has a thickness less than the thickness of the support section.

18. The method of claim 10, wherein the ultrasound transducer probe is positioned near a Postcentral gyrus (poCG) and a Supramarginal gyrus (SMG) of the brain.

19. A functional ultrasound imaging system comprising:
an ultrasound probe including a transducer for functional ultrasound imaging;
a cranial implant including an imaging window constructed of sonolucent material, wherein the functional ultrasound probe is positioned on the imaging window; and
a functional ultrasound controller coupled to the ultrasound probe, the functional ultrasound controller taking functional ultrasound images of a brain via the ultrasound probe.

20. The functional ultrasound imaging system of claim 19, wherein the sonolucent window is fabricated from polymethyl methacrylate (PMMA).
